(19) United States
(12) Patent Application Publication
(10) Pub. No.: US 2018/0296486 A1
(43) Pub. Date: Oct. 18, 2018

(54) FOOD INDEPENDENT IMMEDIATE RELEASE DRUG FORMULATION WITH ABUSE DETERRENCE AND OVERDOSE PROTECTION

(71) Applicant: KASHIV PHARMA, LLC, Bridgewater, NJ (US)

(72) Inventors: Navnit H. Shah, Clifton, NJ (US); Wantanee Phuapradit, Montville, NJ (US); Dipen Desai, Whippany, NJ (US); Siva Ram Kiran Vaka, Piscataway, NJ (US); Kanji Meghpara, Morris Plains, NJ (US); Atsawin Thongsukmak, Piscataway, NJ (US)

(73) Assignee: KASHIV PHARMA, LLC, Bridgewater, NJ (US)

(21) Appl. No.: 15/956,279

(22) Filed: Apr. 18, 2018

Related U.S. Application Data
(60) Provisional application No. 62/486,806, filed on Apr. 18, 2017.

(51) Int. Cl. A61K 9/14; A61K 9/48; A61K 9/20; A61K 31/485

(52) U.S. Cl. CPC A61K 9/146; A61K 9/4808; A61K 9/485; A61K 9/4858; A61K 31/485; A61K 9/2077; A61K 9/2086; A61K 9/2054; A61K 9/2013; A61K 9/4866

(57) ABSTRACT

The presently disclosed subject matter provides a solid immediate release pharmaceutical particulate dosage form containing one population of particulates, and/or a solid immediate release pharmaceutical multi-particulate dosage form containing at least two different populations of particulates. In certain embodiments, the immediate release pharmaceutical dosage forms contain at least three different populations of multi-particulates. Each population of particulates is designed for a specific function to accomplish the desired combination of abuse deterrence and overdose protection.

### CROSS-REFERENCE TO RELATED APPLICATION

The present application claims priority to U.S. Provisional Application No. 62/486,806 filed on Apr. 18, 2017, the content of which is incorporated herein by reference in its entirety.

### 1. FIELD OF THE INVENTION

The present disclosure relates to food independent immediate release pharmaceutical dosage forms with abuse deterrent (AD) and overdose protection (ODP) properties/features, and processes of manufacture.

### 2. BACKGROUND

Governmental reports state that prescription drug abuse is the fastest growing drug problem in the United States, and a survey indicated that nearly one-third of people age 12 and above who used drugs illicitly for the first time in 2009 began by the nonmedical use of a prescription drug. For example, opioid analgesics can be abused by: swallowing whole in excessive quantities; crushing and swallowing; crushing and inhaling nasally (“snorting”); crushing and smoking; or crushing, dissolving, and injecting the prescription drug.

Abuse can also involve some physical or mechanical manipulation of a dosage form so that larger amounts of immediately available drug can be taken orally, nasally, or by intravenous injection. Reports of overdosing and death from prescription pain products rose sharply in the early 2000s. For example, among opioid dosage forms, immediate release oxycodone is the third most prone to overdose.

In March 2016, the FDA published a guidance document describing general procedures for developing and evaluating abuse deterrence of generic solid oral opioid products formulated to incorporate physical or chemical barriers, agonists/antagonists, aversive agents, or combinations of these technologies. The FDA recommends the following evaluations, involving all potential routes of abuse, of the abuse deterrence of generic solid oral opioid drug products:
1. Injection (parenteral route)—evaluate the extractability and syringeability of intact and mechanically manipulated products.
2. Ingestion (oral route)—evaluate extractability, dissolution, and where applicable, the rate and extent of a product's absorption for intact and mechanically or chemically manipulated products.
3. Insufflation (nasal route)—evaluate nasal availability and likability of mechanically manipulated and insufflated products.
4. Smoking (inhalation route)—evaluate the ability to sublimate intact and mechanically or chemically manipulated products.
   FDA further describes mechanical manipulation, with and without thermal pretreatment (e.g., freezing at −20° C.; heating), as involving cutting, grating, and milling.

A few abuse-resistant opioid products are currently approved for marketing, including OXYCONTIN® (oxycodone hydrochloride extended release tablets), XTAMPZA™ ER (oxycodone hydrochloride ER), TARGINIQ® (oxycodone HCl and naloxone HCl), and EMBEDA® (morphine sulfate and naltrexone hydrochloride). Other products, such as OXAYDO® (oxycodone hydrochloride IR tablets), SUBOXONE® (buprenorphine and naloxone) and OPANA ER® (oxymorphone), also purport to have abuse deterrent properties but do not have a formal claim on the label. As noted by FDA in their 2015 guidelines, most abuse-deterrent technologies have not yet proven successful at deterring the most common form of abuse: swallowing a number of intact capsules or tablets.

A need, therefore, remains for improved formulations that make it difficult, if not impossible, for individuals to abuse or misuse opioids, not only by snorting and/or extraction of drug, but also by ingesting multiple doses. New formulations are needed that can be used with pharmaceutical products intended for immediate release. There is also a need for improved formulations that do not compromise/reduce the release of opioids in fed or fasted states, when consumed in amounts effective for the intended therapeutic purpose, while reducing or preventing the effects of overdose, whether intentional or unintentional (e.g., accidental). Such formulations should combine overdose protection and abuse deterrence in a single dosage form and thereby address multiple health-related concerns, especially regarding habit-forming opioid compounds, for which there is a high propensity for abuse and overdose. Such formulations should not compromise/reduce the release of opioids when consumed as intended, and should also reduce or prevent the effects of intentional or unintentional overdose. These dosage forms must also allow the active pharmaceutical ingredient to be soluble in the gastrointestinal tract and have the desired pharmacological activity. In the case of opioids, the pharmacological activity would be, for example, an analgesic effect.

### 3. SUMMARY OF THE INVENTION

The presently disclosed subject matter provides food independent, multiparticulate dosage forms that provide an immediate release of an opioid when a single dosage unit is consumed intact, independent of fed or fasted state of an individual consuming the dosage form, and also provides overdose protection when multiple dosage units are consumed intact. In certain example embodiments, the dosage form includes Active Particulates including a therapeutically effective amount of at least one opioid embedded in a polymer matrix, and an acid labile functional coat; and Triggering Particulates including an alkaline agent. The acid labile functional coat includes at least one functional coat layer FC 1 including at least one acid, a water-insoluble nonionic polymer, and a base polymer that is at least partially neutralized as a cationic salt at gastric fluid pH. The alkaline agent is present in an amount sufficient, when three or more dosage units are consumed together, to increase gastric fluid pH to a level that reduces the solubility of the acid labile functional coat and causes a decrease in the immediate release of the opioid from the dosage form to provide the overdose protection. The acid is present in an amount that keeps the base polymer in partially neutralized form and maintains immediate release properties of the dosage form in the fed state.

In certain embodiments, the partially neutralized base polymer includes a copolymer of dimethyl aminoethyl methacrylate, butyl methacrylate, and methyl methacrylate.

In certain embodiments, the acid is selected from the group consisting of succinic acid, hydrochloric acid, sulfuric acid, nitric acid, lactic acid, phosphoric acid, citric acid, acetic acid, malic acid, fumaric acid, stearic acid, tartaric acid, boric acid, benzoic acid, and mixtures thereof.

In certain embodiments, the acid is present in an amount of between about 0.1% w/w and about 5% w/w of the dosage form. In certain embodiments, the acid is present in an amount of between about 0.1% w/w and about 0.25% w/w of the dosage form. In certain embodiments, the acid can be succinic acid.

In certain embodiments, the dosage form can include a second functional coat layer FC 2, completely or partially surrounding FC 1.

In certain embodiments, the water-insoluble nonionic polymer includes cellulose acetate; cellulose acetate-based polymers; polyvinyl acetate polymers; polyvinyl acetate-based copolymers; ethylcellulose; methacrylic acid and methyl methacrylate (1:1); methacrylic acid and methyl methacrylate (1:2); copolymers of ethyl acrylate and methyl methacrylate; or mixtures thereof.

In certain embodiments, the water-insoluble nonionic polymer can be cellulose acetate.

In certain embodiments, the partially neutralized base polymer and the water-insoluble nonionic polymer can be present in a weight ratio of about 50:50.

In certain embodiments, FC 2 includes an acid and a base polymer that is at least partially neutralized as a cationic salt at gastric fluid pH.

In certain embodiments, the partially neutralized base polymer of FC 2 can include a copolymer of dimethylaminoethyl methacrylate, butyl methacrylate, and methyl methacrylate.

In certain embodiments, the polymer matrix includes a nonionic polymer selected from the group consisting of a copolymer of ethyl acrylate, methyl methacrylate, and a low content of methacrylic acid ester with quaternary ammonium groups; hydroxypropylcellulose; hydroxypropyl methylcellulose; hydroxyethylcellulose; ethylcellulose; cellulose acetate butyrate; cellulose acetate; polyvinyl acetate-based polymers; polyethylene oxide polymers; and mixtures thereof.

In certain embodiments, the nonionic polymer includes a mixture of a polyethylene oxide polymer, hydroxypropyl methylcellulose, and a polyvinyl acetate-based polymer.

In certain embodiments, the nonionic polymer includes a mixture of a polyethylene oxide polymer and hydroxypropyl methylcellulose.

In certain embodiments, the alkaline agent present in the Triggering Particulates can be selected from the group consisting of aluminum hydroxide, sodium hydroxide, potassium hydroxide, calcium hydroxide, magnesium hydroxide, calcium carbonate, sodium carbonate, potassium bicarbonate, sodium bicarbonate, ammonia, tertiary sodium phosphate, diethanolamine, ethylenediamine, N-methylglucamine, L-lysine, and mixtures thereof.

In certain embodiments, the alkaline agent includes magnesium hydroxide.

In certain embodiments, the alkaline agent can be present in an amount of up to about 40% w/w of the total weight of the dosage form.

In certain embodiments, the alkaline agent can be present in an amount of from about 25% w/w to about 32% w/w of the total weight of the dosage form.

In certain embodiments, the Active Particulates can include a plasticizer in an amount sufficient to enhance elasticity and crush resistance of the polymer matrix.

In certain embodiments, the crush resistance of the polymer matrix can be enhanced to an extent that prevents reducing particulates to a size that can be insufflated.

In certain embodiments, the plasticizer can act as one or more of an aversion agent and a tissue irritant.

In certain embodiments, the plasticizer can be selected from the group consisting of triethyl citrate, propylene glycol, polyethylene glycols, triacetin, diethylene glycol monoethyl ether, dibutyl sebacate, diethyl phthalate, and mixtures thereof.

In certain embodiments, the Active Particulates can include one or more of a surfactant and a viscosity enhancing agent.

In certain embodiments, the opioid can be selected from the group consisting of oxycodone, hydrocodone, oxymorphone, and hydromorphone, and pharmaceutically acceptable salts thereof.

The presently disclosed subject matter also provides methods of treating pain. In an example embodiment, the method includes administering to a patient in need thereof a food independent, multiparticulate dosage form that provides an immediate release of an opioid when a single dosage unit is consumed intact, independent of fed or fasted state of an individual consuming the dosage form. In certain embodiments, the food independent, multiparticulate dosage form also provides overdose protection when multiple dosage units are consumed intact.

In certain embodiments, the food independent, multiparticulate dosage form includes Active Particulates including a therapeutically effective amount of at least one opioid embedded in a polymer matrix, and an acid labile functional coat; and Triggering Particulates including an alkaline agent.

In certain embodiments, the acid labile functional coat includes at least one functional coat layer FC 1 including at least one acid, a water-insoluble nonionic polymer, and a base polymer that is at least partially neutralized as a cationic salt at gastric fluid pH.

In certain embodiments, the alkaline agent can be present in an amount sufficient, when three or more dosage units are consumed together, to increase gastric fluid pH to a level that reduces the solubility of the acid labile functional coat and causes a decrease in the immediate release of the opioid from the dosage form to provide the overdose protection.

In certain embodiments, the acid can be present in an amount that keeps the base polymer in partially neutralized form in fed state, maintaining the immediate release properties of the dosage form.

The presently disclosed subject matter also provides methods of making a food independent, multiparticulate dosage form that provides an immediate release of an opioid when a single dosage unit is consumed intact, independent of fed or fasted state of an individual consuming the dosage form, and provides overdose protection when multiple dosage units are consumed intact. In an example embodiment, the method includes making Active Particulates by hot-melt extruding a blend of oxycodone hydrochloride, polyethylene oxide, and at least one additional water-soluble nonionic polymer, and coating the extrudates with an acid labile functional coat including at least one functional coat layer FC 1 including at least one acid, a water-insoluble nonionic polymer, and a base polymer that is at least partially neutralized as a cationic salt at gastric fluid pH; making Triggering Particulates including an alkaline agent; mixing the Active Particulates and the Triggering Particulates into a uniform blend; mixing the blend with magnesium stearate; and compressing the mixture into a tablet.

### 4. BRIEF DESCRIPTION OF THE DRAWINGS

FIG. 1 depicts a schematic representation of an Active Particulate according to certain embodiments.

FIG. 2 compares in vitro release profiles of oxycodone hydrochloride tablets with (Test Product B) and without (Test Product A) succinic acid, at pH 5.5.

### 5. DETAILED DESCRIPTION

To date, there remains a need for improved immediate release pharmaceutical dosage forms that make it difficult, if not impossible, for individuals to take the dosage forms in a manner other than that intended by the manufacturer. In certain embodiments, the present disclosure provides improved solid oral immediate release pharmaceutical particulate and multi-particulate dosage forms containing at least one population of particulates, e.g., particulates comprising an active agent (e.g., an opioid). In certain embodiments, the present disclosure provides improved solid oral immediate release pharmaceutical multi-particulate dosage forms containing at least two populations of particulates, e.g., (1) Active Particulates containing an opioid, and (2) Triggering Particulates containing an alkaline agent and/or a pH-stabilizing agent. In certain embodiments, the immediate release pharmaceutical multi-particulate dosage forms contain at least three different populations of particulates. In certain embodiments, the immediate release pharmaceutical multi-particulate dosage forms contain at least four, at least five, or at least six or more different populations of particulates. In certain embodiments, the Active Particulates comprise an opioid(s), alkaline agent(s), and/or a pH-stabilizing agent(s); in certain embodiments, the alkaline agent(s) and/or pH-stabilizing agent(s) can be covering/surrounding the Active Particulates; in certain embodiments, the alkaline agent is present in Triggering Particulates. Each population of particulates is designed for a specific function to accomplish the desired combination of abuse deterrence and overdose protection.

In certain embodiments, the immediate release pharmaceutical dosage forms contain an Active Particulate population, which is a crush-resistant population of particulates comprising an active agent and at least a first functional coat layer (e.g., FC 1) comprising a cationic polymer and at least one acid, wherein the functional coat allows the release of the active agent in an aqueous or nonaqueous environment with a pH of up to about 5. This feature of the functional coat layer results in reduction/prevention/slowing of release at a pH above about 5 to provide overdose protection (ODP). In certain embodiments, the release rate of the active agent (e.g., an opioid) is reduced in the presence of food, e.g., in fed state. In certain embodiments, the presence of food raises the gastric fluid pH to about 4.5-5, and such increase in pH neutralizes the cationic polymer into a free base, reducing the release rate of active agent from the dosage form. In certain embodiments, the presence of an acid in the functional coat layer at least partially neutralizes the free base polymer formed at elevated pH. In certain embodiments, presence of partially neutralized base polymer maintains immediate release properties of the dosage form in fed state.

In certain embodiments, the Active Particulates can further include a functional coat layer (e.g., FC 2) on top of FC 1. In certain embodiments, the Active Particulates can include an additional functional coat layer (e.g., FC 0) between the seal coat (or the core) and FC 1 (FC 0, FC 1, and FC 2 are described in detail herein). In certain embodiments, FC 0 and FC 2 can further enhance the ODP features of the Active Particulates in the event of an overdose (e.g., administration/consumption of three or more dosage units). In certain embodiments, FC 0 and/or FC 2 aid FC 1 in preventing or slowing release of the active agent from the Active Particulate in an aqueous or nonaqueous environment with a pH above about 5. In certain embodiments, the Active Particulates can further include an over coat that aids in maintaining the retarded release of active agent when three or more dosage units are consumed. In certain embodiments, the over coat prevents/reduces the interaction of EUDRAGIT® E PO present in functional coat layer(s) (e.g., FC 1, or, when present, FC 2) with the alkaline agent present in the dosage form to maintain the retarded release of the active agent when three or more dosage units are consumed.

In certain embodiments, Active Particulates contain an opioid(s) as the active agent (Opioid Particulates).

In certain embodiments, the dosage form contains a Triggering Particulate containing an alkaline agent that increases the pH of the aqueous or nonaqueous solution to above about pH 5 in the presence of three or more dosage units. In certain embodiments, the Triggering Particulates do not alter the pH of GI fluid when one or two dosage units are consumed as intended. The Triggering Particulate can also contain a pH-stabilizing agent that maintains the increased pH above about 5 for up to five minutes, up to ten minutes, up to 15 minutes, up to 30 minutes, up to 45 minutes, up to one hour, up to 1.5 hours, or up to two hours or more. In certain embodiments, the increase in pH above about 5 reduces the dissolution of the functional coat (e.g., one or more functional coat layers), and thereby prevents or slows the release of the active agent from the Active Particulates. In certain embodiments, the Triggering Particulates do not include any opioid. In certain embodiments, the Triggering Particulates are crush-resistant.

In certain embodiments, the immediate release pharmaceutical dosage forms comprise a Viscosity Enhancing Particulate population containing a viscosity-building polymer(s) that increases the viscosity of the aqueous or nonaqueous solution if tampered with or taken in doses above those prescribed or in a manner inconsistent with the manufacturer's instructions. In certain embodiments, the Viscosity Enhancing Particulates do not include any opioid. In certain embodiments, the Viscosity Enhancing Particulates are crush-resistant.

In certain embodiments, the pharmaceutical compounds for use in the present disclosure are those at risk for accidental (e.g., unintentional) or intentional overdose via, for example, the oral route, or misuse via, for example, the oral/intravenous/nasal/smoking routes. In certain embodiments, the active agent is an opioid.

The presently disclosed subject matter provides abuse deterrent and/or overdose-resistant immediate release pharmaceutical dosage forms that do not compromise/reduce the release of opioids when consumed in amounts effective for intended therapeutic purpose, regardless of fed or fasted conditions, while providing overdose protection when three or more dosage units are consumed, also regardless of fed or fasted conditions. The dosage forms of the disclosure comprise particulate dosage forms, or multi-particulate dosage forms containing at least two different populations of particulates.

In certain embodiments, included in the scope of the disclosure is a solid immediate release (IR) multi-particulate dosage form with abuse deterrent and overdose protection properties comprising a first population of particulates (Active Particulates) comprising a therapeutically effective amount of at least one active agent (e.g., an opioid) embedded in a polymer matrix, a functional coat comprising one or more functional coat layers (e.g., FC 1), and an over coat. In certain embodiments, additional optional functional coat layers (e.g., FC 0 and/or FC 2) are included in the functional coat of the Active Particulates. In certain embodiments, the FC 1 layer comprises a water-insoluble pH-independent polymer (e.g., a water-insoluble nonionic polymer) insoluble in physiological fluids and/or organic solvents, and a cationic pH-dependent polymer (a base polymer completely or partially neutralized as a cationic salt) that dissolves and acts as a pore former at a pH of less than about 5.0. In certain embodiments, the over coat comprises a nonionic water-soluble polymer. In certain embodiments, a second population of particulates comprises an alkaline agent. In certain embodiments, the second population of particulates comprises an alkaline agent and a pH-stabilizing agent. In certain embodiments, the alkaline agent raises the gastric pH when three or more dosage units are ingested, and the pH-stabilizing agent maintains the elevated pH for a finite time.

In certain embodiments, the overdose protection (ODP) properties comprise reduction in abuse potential by, for example, orally ingesting three or more intact tablets together.

In certain embodiments, the ODP properties comprise reduction in opioid release to less than about 50% at 30 minutes when three or more units of the dosage form are consumed.

In certain embodiments, the abuse deterrent properties comprise resistance to syringeability, wherein less than 10% of the opioid is available in a syringeable form, e.g., less than 10% of the opioid provided in a dosage form can be extracted, after grinding or crushing followed by dissolution/suspension in a liquid, as a syringeable liquid.

In certain embodiments, abuse deterrent properties comprise resistance to grinding/crushing, wherein grinding or crushing of the dosage form provides more than 50% of particulates in the size range of 250-500 μm or greater.

In certain embodiments, the abuse deterrent elements enhance the ODP properties of the dosage form.

In certain embodiments, the ODP elements enhance abuse deterrent properties of the dosage form.

### 5.1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in the specification and/or in conjunction with the term “comprising” in the claims can mean “one,” but it is also consistent with the meaning of “one or more,” “at least one,” and “one or more than one.” Still further, the terms “having,” “including,” “containing” and “comprising” are interchangeable, and one of skill in the art is cognizant that these terms are open-ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 15%, up to 10%, up to 5%, or up to 1% of a given value. Alternatively, particularly with respect to biological systems or processes, the term can mean within an order of magnitude, preferably within five-fold, and more preferably within two-fold, of a value.

The term “active agent,” “drug,” “compound,” “active pharmaceutical ingredient,” or “API” refers to a pharmaceutically active substance which includes, without limitation, drugs susceptible to abuse and/or overdose. In certain embodiments, the active agent is an opioid analgesic.

The term “opioid” or “opioid analgesic” includes single compounds and a mixture of compounds selected from the group of opioids that provide, e.g., an analgesic effect. For example, opioids can include, without limitation, an opioid agonist, a mixed opioid agonist-antagonist, or a partial opioid agonist. In certain embodiments, the opioid can be a stereoisomer, ether, salt, hydrate or solvate thereof. The terms opioid and opioid analgesic are also meant to encompass the use of all such possible forms as well as their racemic and resolved forms thereof, and all tautomers as well. The term “racemic” refers to a mixture of equal parts of enantiomers.

The term “immediate release” or “IR” refers to dosage forms that are formulated to allow the drug to dissolve in the gastrointestinal contents/fluids with no intention of delaying or prolonging the dissolution or absorption of the drug when taken as prescribed or in a manner consistent with manufacturer's instructions.

The term “extended release” or “ER” refers to dosage forms that are formulated to allow the drug to be available over a greater period of time after administration, thereby allowing a reduction in dosing frequency, as compared to a drug presented as a conventional dosage form (e.g., immediate release).

The term “food independent,” as used herein, refers to oral, immediate release dosage forms for which the release and/or the rate of release of the active agent from the dosage form is not significantly affected/altered (e.g., not significantly increased or decreased) by the fed or fasted state of the individual consuming the dosage form, i.e., the release and/or the rate of release of the active agent is independent of the fed or fasted state of the individual. Thus, the efficacy of the dosage form is not compromised by the fed or fasted state of the individual consuming the dosage form.

The term “particulate” refers to a discrete, small, repetitive unit of particles, granules, or pellets that include at least one excipient and, optionally, an active agent (e.g., an opioid).

The term “multi-particulate” refers to at least two different populations of particulates.

The term “dosage form” refers to an oral particulate solid drug delivery system that, in the present technology, includes at least one or two populations of particulates.

The term “dosage unit” refers to a single tablet (e.g., tablet, tablet-in-tablet, bilayer tablet, multilayer tablet, etc.), capsule, pill, or other solid dosage form.

The term “coat” refers to a coating, layer, membrane, film, etc. applied to a surface, and, in certain embodiments, can partially, substantially, or completely surround, envelop, cover, enclose, or encase the surface of a particulate, granule, pellet, drug, dosage unit, or the like to which it is applied. For example, a coat can cover portions of the surface to which it is applied, e.g., as a partial layer, partial coating, partial membrane, or partial film, or the coat can completely cover the surface to which it is applied.

The terms “acid labile coat” or “functional coat” (or “coatings”) refer to a coat comprising a component(s) that will dissolve or degrade (partially or completely) in an acidic environment (e.g., in a solution with an acidic pH). In certain embodiments, the acidic pH can be, for example, below about 7.0, below about 6.0, below about 5.0, below about 4.0, below about 3.0, or below about 2.0, or below about 1.0. Typically, the pH at which an acid labile coat/functional coat of the present disclosure will dissolve is in the normal physiological pH (e.g., the range of normal physiological pH values) of the stomach, such as from about 1.0 to about 5.0, from about 1.0 to about 4.0, or from about 2.0 to about 3.0. Typically, the acid labile coat/functional coat dissolves or degrades more slowly, or to only a small extent, when present in a solution with a pH that is considered not acidic (e.g., nonacidic and/or less acidic; e.g., at a pH above about 5.0, above about 6.0, or above about 7.0). It will be understood that the acid labile coat/functional coat can be prepared and designed to dissolve or degrade (partially or substantially) within any desired pH range, and to not dissolve or degrade (partially or substantially) within any desired pH range. For example, the acid labile coat/functional coat can be designed to dissolve at any pH, e.g., below about 5.0; above that level, dissolution is inhibited, reduced or slowed. As the pH increases, the dissolution/degradation can slow further, and can stop nearly completely. The acid labile coat/functional coat affects the rate of release, in vitro or in vivo, of an active drug(s), e.g., an opioid(s). Such coatings or coats are sometimes referred to as “rate-limiting” or “rate-controlling”; the particular polymer(s) responsible for affecting the rate of release in the coating or coat can also be referred to as “rate-limiting” or “rate-controlling.” An acid labile coat/functional coat can comprise one or more functional coat layers.

The term “alkaline agent” can be used to refer to an excipient that acts to increase the pH of, e.g., the gastric fluid (e.g., roughly pH 1.2-4.5) to a pH greater than about 5.0. For example, alkaline agent can refer to substances that can increase the pH to greater than 4.5, greater than 5.0, greater than 5.5, etc. It also refers to basic substances and substances that can convert an acidic environment to a less acidic or a basic environment. Typically, these agents, when present in a sufficient amount, can raise the pH of the stomach to beyond physiological levels and thereby prevent, reduce, or inhibit dissolution of an acid labile substance or coat. Examples of alkaline agents include: aluminum hydroxide, sodium hydroxide, potassium hydroxide, calcium hydroxide, magnesium hydroxide, aluminum oxide, sodium oxide, potassium oxide, calcium oxide, magnesium oxide, calcium carbonate, sodium carbonate, potassium bicarbonate, sodium bicarbonate, ammonia, tertiary sodium phosphate, diethanolamine, ethylenediamine, N-methylglucamine, L-lysine, and combinations thereof.

The term “pH-stabilizing agent” refers to salts of weak acids/weak bases that act to maintain or stabilize the elevated pH of aqueous or nonaqueous solutions/gastric fluid caused by the alkaline agent. For example, a pH-stabilizing agent maintains the pH of the gastric fluid at a pH greater than 5.0 for a finite time.

The term “viscosity-building polymer” as used herein refers to a polymer or group of polymers that increase the viscosity of a solution/gastric fluid if the dosage form is tampered with or taken in doses above those prescribed, or in some other manner inconsistent with the manufacturer's instructions.

The term “nonionic polymer” refers to a nonionic pH-independent polymer that cannot be changed to any ionic form/salt in presence of an acid or a base.

The term “water-insoluble polymer” refers to a polymer generally insoluble in water, physiological fluids, and ethanol. As used herein, the term “water-insoluble polymer” includes nonionic and anionic polymers.

The term “water-insoluble nonionic polymer” refers to a nonionic pH-independent polymer generally insoluble in water, physiological fluids, and ethanol.

The term “water-soluble nonionic polymer” refers to a nonionic pH-independent polymer generally soluble in water, physiological fluids, and ethanol.

The term “cationic polymer” refers to a pH-dependent base polymer, a pH-dependent base polymer partially neutralized with an acid, or a pH dependent base polymer completely neutralized with an acid. The term “cationic polymer” can include a base polymer that is completely or partially neutralized as a cationic salt, and is generally soluble in an acidic pH range, e.g., gastric fluid or simulated gastric fluid (SGF).

The term “mini-tablet” refers to a tablet with a diameter equal to or smaller than 4 mm. They can be filled into a capsule or compressed into a larger tablet.

The term “abuse-deterrent formulation,” “abuse-deterrent composition,” “abuse-resistant formulation,” “abuse-resistant composition,” or “ADF” are used interchangeably to refer to a dosage form that reduces the potential for abuse but delivers a therapeutically effective dose when administered as directed. For example, these terms refer to a dosage form that can be at least resistant, with or without heat treatment or freezing, to crushing, grinding, melting, cutting, extracting, dose dumping (e.g., alcohol dose dumping), and solubilizing for injection purposes. Improper administration includes, without limitation, tampering with the dosage form and/or administering the drug by any route other than that instructed. For example, and without limitation, improper administration includes snorting after grinding, administration after heat treatment, oral administration after crushing, or parenteral administration after extraction with a solvent such as water, ethanol, isopropanol, acetone, acetic acid, vinegar, carbonated beverages, and the like, and combinations thereof.

The term “abuse” means the intentional, nontherapeutic use of a dosage form or active agent, to achieve a desirable psychological or physiological effect. For example, these terms refer to tampering with the dosage form and/or administering the drug in a manner inconsistent with the manufacturer's instructions. Methods of tampering or abuse include, but are not limited to, crushing, grinding, melting, cutting, heating, freezing, extracting, dose dumping, and solubilizing for injection purposes.

The term “in a manner inconsistent with the manufacturer's instructions” is meant to include, but is not limited to, consuming amounts greater than amounts described on the label or prescribed by a licensed physician, and/or altering by any means (e.g., crushing, breaking, milling, melting, separating, etc.) the dosage forms such that the active agent can be crushed, ground, melted, cut, extracted, dose dumped (e.g., alcohol dose dumping), and/or solubilized for injection purposes.

The term “syringeability” refers, for example, to the ability of an agent (e.g., an opioid) to be extracted from a product formulation or dosage form into a syringe, i.e., the agent is in a syringeable form. For example, a solid dosage form can be dissolved/suspended in water, and an agent present in the dosage form can be extracted from the resulting liquid into a syringe in the form of a syringeable liquid.

The term “available in syringeable form,” as used herein, refers to availability of an agent (e.g., an opioid) to be extracted into a syringe from a solution/suspension of a solid dosage form. The amount or percentage of such extracted agent could be termed as the amount or percentage available in syringeable form, or available as a syringeable liquid, or the like.

The term “crush resistant” or “resistant to crushing” means, for example, a granule or particulate (e.g., an Active Granule) that can deform but does not break into powder form when pressure greater than 500 N is applied, when using a suitable hardness tester. Such resistance to crushing deters the abuse of the dosage form.

The term “grinding” refers to a process of reducing, or attempting to reduce, one or more tablets into small fragments, e.g., in the form of powder, following a specific grinding pattern (e.g., two minutes grinding/one minute rest/two minutes grinding) using, for example, an electrical grinding means (e.g., coffee grinder or IKA laboratory grinder).

The term “resistant to alcohol extraction” is used to refer to two or more dosage units (e.g., any form of tablets or capsules) that at least fulfill the condition that in vitro dissolution, characterized by the percentage of active agent released at, e.g., 30 minutes or 60 minutes of dissolution, when measured in a USP Apparatus 1 (basket) at 100 rpm in 900 ml simulated gastric fluid comprising 40% ethanol at 37° C., deviates no more than 20% from the corresponding in vitro dissolution measured at the same time point in the same apparatus at the same speed in 900 ml SGF without ethanol at 37° C.

The term “overdose protection” or “ODP” refers to an oral dosage form that reduces the potential for overdose but delivers a therapeutically effective dose when administered as directed or ordered by a licensed physician.

The term “overdose” refers to the administration of the dosage form in amounts or doses above those considered therapeutic (e.g., three or more dosage units; more than two dosage units); in a manner inconsistent with manufacturer's instructions; or in a manner not prescribed. Overdose can be intentional or unintentional (e.g., accidental).

As used herein, use of phrases such as “decreased,” “reduced,” “diminished,” or “lowered” is meant to include at least a 10% change in, e.g., the release of an active agent, with greater percentage changes being preferred for reduction in abuse potential and overdose potential. For example, but without limitation, the change can be greater than 25%, 35%, 45%, 55%, 65%, 75%, 85%, 95%, 96%, 97%, 98%, 99%, or increments therein.

### 5.2. Active Particulates

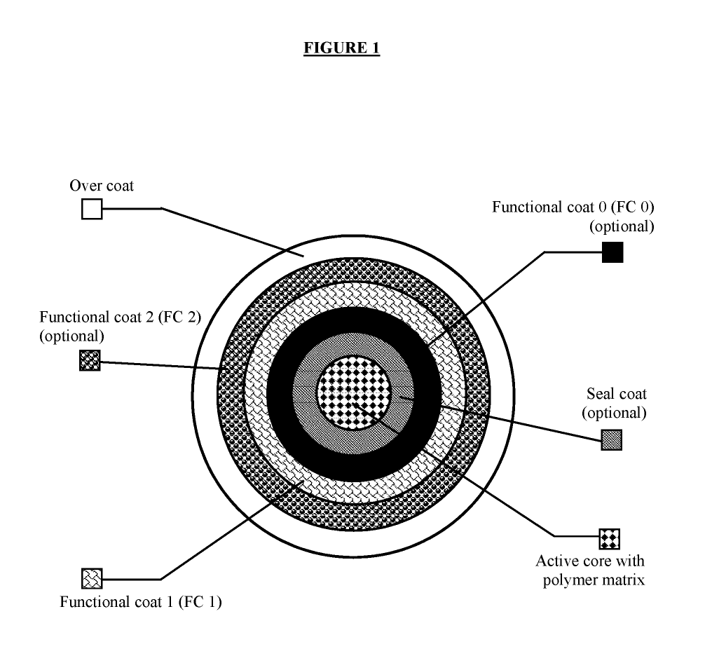

With reference to FIG. 1 for the purpose of illustration and not limitation, there is provided a schematic illustrating functional coat layers FC 0, FC 1, and FC 2; an active core with a polymer matrix; an over coat; and a seal coat.

The Active Particulates contain the active agent. In certain embodiments, the Active Particulates can include a polymer matrix that in some embodiments can include an active agent, a hydrophilic polyethylene oxide (PEO) polymer, a cationic and/or a nonionic polymer, an antioxidant, a plasticizer, and/or a surfactant. The polymer matrix of Active Particulates (e.g., Active Granules) containing the active agent can be directly coated/surrounded by a seal coat. In certain embodiments, the seal coat can be made with a water-soluble nonionic polymer. In certain embodiments, the seal coat is optional. In certain embodiments, the polymer matrix core (in absence of a seal coat)), or the seal coat (when present over the polymer matrix core) is surrounded by one or more functional coat layers (e.g., FC 0, FC 1, FC 2). In certain embodiments, the polymer matrix, or the seal coat covering the polymer matrix is directly covered by at least one functional coat layer (e.g., FC 1). In certain embodiments, one or more functional coat layers can include a water-insoluble nonionic polymer, as well as a cationic polymer that behaves as a pore former at pH below about 5.0. In certain embodiments, the Active Particulates comprising FC 1 can further comprise FC 0, located between the polymer matrix (or seal coat) and FC 1. In certain embodiments, the Active Particulates comprising FC 1 can further comprise FC 2, coated over FC 1. In certain embodiments, FC 0 and/or FC 2 contain a cationic polymer (e.g., a base polymer completely or partially neutralized with an acid) and, optionally, a water-insoluble nonionic polymer. In certain embodiments, the Active Particulates further include an over coat that contains a water-soluble nonionic polymer and partially or completely surrounds the outermost functional coat layer.

In certain embodiments of Active Particulates, each of FC 0, FC 1, and/or FC 2 accomplishes the role of overdose protection coupled with an alkaline agent and, optionally, a pH-stabilizing agent present in the dosage form (tablets, capsules, etc.). In certain embodiments, FC 0 and/or FC 2 can provide enhanced ODP, in addition to that provided by FC 1, when coupled with the alkaline agent and/or pH-stabilizing agent contained in the Triggering Particulates. In certain embodiments, the release of opioid from the dosage form of the disclosure is independent of the fed or fasted state of the subject or patient. In certain embodiments, the immediate release pharmaceutical dosage forms of the disclosure do not affect the release of opioids, when consumed either in fed or fasted state, in amounts effective for intended therapeutic purpose, while providing overdose protection when three or more dosage units are consumed.

### 5.2.1. Active Agents

In certain embodiments, the Active Particulates contain at least one active agent, e.g., an opioid. In certain embodiments, different populations of Active Particulates contain different active agents.

The Active Particulates can be coated with at least one functional coat layer (e.g., FC 1). In certain embodiments, FC 1 includes a polymer (e.g., a nonionic polymer) that is insoluble in water, and a cationic polymer (e.g., a base polymer completely or partially neutralized with an acid) that behaves as a pore former at a pH of less than about 5 and is insoluble in fluids with a pH above about 5 (e.g., at a pH of about 5 or greater). Surprisingly, it has been found that a functional coat (e.g., at least one functional coat layer present in Active Particulates) containing a base polymer partially neutralized with an acid, provides a therapeutically acceptable immediate release of, e.g., an opioid, in fed as well as fasted states, and the amount of alkaline agent (e.g., magnesium hydroxide) in the dosage form does not affect the release of opioid from the dosage form, when taken in a manner consistent with manufacturer's instructions, or in a manner prescribed (e.g., one or two dosage units are taken as intended). It has been found that the amount of alkaline agent (e.g., magnesium hydroxide), e.g., about 25-30% w/w of the dosage form, does not alter the pH of the GI fluid in the fed or fasted state, or affect the release of opioid from the dosage form, when one or two dosage units are consumed as intended. The partially neutralized base polymer in the functional coat layer of the Active Particulates and the amount of alkaline agent in the dosage form provide an intended immediate release of the opioid from the dosage form, independent of the fed or fasted state of the subject or patient, when consumed as intended, and provide protection from the effects of overdose when three or more dosage units are consumed.

In certain embodiments, the pharmaceutically active agent is present in the dosage form in an amount effective for the intended therapeutic purpose. These amounts are well known in the art. Indeed, the doses at which any of the presently known active agents embraced/contemplated by the present disclosure can be given safely and effectively for the intended therapeutic purpose are known to those of skill in the art. In certain embodiments, the active agent (e.g., an opioid) is present in an amount of about 0.1% to about 95% w/w of the Active Particulate before the addition of the (optional) seal coat, or any functional coat layer(s) (i.e., about 0.1% to about 95% w/w of the polymer matrix embedded with active agent). In certain embodiments, the active agent is present in an amount of about 0.2% to about 90%, about 0.3% to about 85%, about 0.4% to about 80%, about 0.5% to about 75%, about 0.6% to about 70%, about 0.7% to about 65%, about 0.8% to about 60%, about 0.9% to about 55%, about 1% to about 50%, about 2.5% to about 45%, about 5% to about 40%, about 7.5% to about 35%, about 10% to about 30%, about 12.5% to about 25%, or about 15% to about 20% w/w of the polymer matrix embedded with active agent. In certain embodiments, the active agent (e.g., opioid) is present in an amount of at least about 0.1%, 0.2%, 0.5%, 1%, 5%, 10%, 15%, 20%, 25%, 30%, 35%, 40%, 45%, 50%, 55%, 60%, 65%, 70%, 75%, 80%, 85%, 90%, or 95% w/w of the polymer matrix embedded with active agent, or intermediate values thereof.

In certain embodiments, the active agents are drugs prone to abuse, misuse, and/or overdose. In certain embodiments, the active agents can include, without limitation, members of the therapeutic categories such as analgesics, anti-inflammatory agents, anthelmintics, anti-arrhythmic agents, anti-bacterial agents, anti-viral agents, anticoagulants, anti-depressants, anti-diabetic agents, anti-epileptic agents, anti-fungal agents, anti-gout agents, anti-hypertensive agents, anti-malarial agents, anti-migraine agents, anti-muscarinic agents, anti-neoplastic agents, erectile dysfunction improving agents, immunosuppressants, anti-protozoa agents, anti-thyroid agents, anti-anxiolytic agents, sedatives, hypnotics, neuroleptics, β-blockers, cardiac inotropic agents, corticosteroids, diuretics, anti-Parkinsonian agents, gastrointestinal agents, histamine receptor antagonists, keratolytics, lipid-regulating agents, anti-angina agents, cox-2 inhibitors, leukotriene inhibitors, macrolides, muscle relaxants, nutritional agents, protease inhibitors, sex hormones, stimulants, anti-osteoporosis agents, anti-obesity agents, cognition enhancers, anti-urinary incontinence agents, nutritional oils, anti-benign prostate hypertrophy agents, essential fatty acids, nonessential fatty acids, and any combinations of two or more thereof.

In certain embodiments, the active agent can be an opioid (e.g., an opioid analgesic). For example, without limitation, the opioid can be alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, hydrocodone, hydromorphone, hydromorphodone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, pantopon, papaveretum, paregoric, pentazocine, phenadoxone, phendimetrazine, phendimetrazone, phenomorphan, phenazocine, phenoperidine, piminodine, piritramide, propheptazine, promedol, properidine, propoxyphene, propylhexedrine, sufentanil, tapentadol, tilidine, tramadol, pharmaceutically acceptable salts thereof.

In certain embodiments, the opioid can be oxycodone, hydrocodone, tapentadol, codeine, oxymorphone, hydromorphone, or pharmaceutically acceptable salts thereof. In certain embodiments, the opioid is oxycodone, hydrocodone, oxymorphone, hydromorphone, or codeine. In certain embodiments, the opioid is a pharmaceutically active salt of oxycodone, hydrocodone, oxymorphone, hydromorphone, or codeine. See, e.g., International Published Application WO 2017/059374, incorporated-by-reference in its entirety herein.

In certain embodiments, the active agents can include, but are not limited to, benzodiazepines (e.g., bromazepam, chlordiazepoxide, clorazepate, diazepam, estazolam, flurazepam, halazepam, ketazolam, lorazepam, nitrazepam, oxazepam, prazepam, quazepam, temazepam, triazolam), barbiturates (e.g., amobarbital, aprobarbotal, butabarbital, butalbital, methohexital, mephobarbital, metharbital, pentobarbital, phenobarbital, secobarbital), and stimulants, such as amphetamines (e.g., amphetamine, dextroamphetamine resin complex, dextroamphetamine, methamphetamine, methylphenidate), as well as dronabinol, glutethimide, methylprylon, ethchlorovynol, ethinamate, fenfluramine, meprobamate, pemoline, levomethadyl, benzphetamine, chlorphentermine, diethylpropion, phentermine, mebutamate, chlortermine, phenylacetone, dronabinol, nabilone, chloral hydrate, ethchlorovynol, paraldehyde, midazolam, and dextropropoxyphene, or pharmaceutically acceptable salts thereof.

Examples of pharmaceutically acceptable salt include, but are not limited to, citrate, oxalate, acetate, maleate, malonate, fumarate, succinate, tosylate, mesylate, hydrochloride, hydrobromide, sulfate, phosphate, methanesulfonate, toluenesulfonate or mixtures and/or forms thereof. Additional pharmaceutically acceptable salts can be found in P. H. Stahl and C. G. Wermuth, editors, Handbook of Pharmaceutical Salts: Properties, Selection and Use, Weinheim/Zürich: Wiley-VCH/VHCA, 2002.

### 5.2.2. Formulation of Active Particulates

In certain embodiments, the Active Particulates (e.g., Active Granules) include an active agent, and a polymer matrix that in some embodiments can include hydrophilic polyoxyethylene (PEO) polymer, a cationic polymer and/or a nonionic polymer, an antioxidant, a plasticizer, and a surfactant. In certain embodiments, the Active Particulates can include a seal coat and at least one functional coat layer (e.g., FC 1). In certain embodiments, the seal coat is optional. In certain embodiments, Active Particulates containing, e.g., FC 1 can further include FC 0 between the polymer matrix and FC 1. In certain embodiments, the Active Particulates include FC 2 over FC 1. In certain embodiments, the Active Particulates include an over coat, comprising a water-soluble nonionic polymer, surrounding the outermost functional coat layer. In certain embodiments, at least one of FC 0, FC 1, and FC 2 includes a water-insoluble nonionic polymer (e.g., generally not soluble in physiological fluids and commonly used organic solvents such as ethanol), and a cationic polymer. The latter behaves as a pore former at a pH below about 5.0, but can swell and become partially permeable, e.g., semipermeable, at a pH above 5.0 (e.g., in intestinal fluids, or in gastric fluids with an elevated pH), thereby substantially preventing release of the active agent (e.g., an opioid) at higher pH. In certain embodiments, the presence of a base polymer partially neutralized with an acid, e.g., succinic acid, maintains a required permeability of the functional coat layer (when one or two dosage units are consumed, as prescribed), for an immediate release of opioid in the GI environment, regardless of whether the subject or patient is in a fed or fasted state.

In certain embodiments, the acids useful for completely or partially neutralizing a base polymer include, but are not limited to, succinic acid, hydrochloric acid, sulfuric acid, nitric acid, lactic acid, phosphoric acid, citric acid, acetic acid, malic acid, fumaric acid, stearic acid, tartaric acid, boric acid, and benzoic acid. In certain embodiments, combinations of acids can be used, including combinations of the above listed acids. In certain embodiments, the amount of acid used to completely or partially neutralize the base polymer can depend upon the strength of the acid used. In certain embodiments, about 0.1% to about 20% acid is used, depending upon the strength of the acid (e.g., stronger acids can be used in lower percentages, and weaker acids can be used in higher percentages). In certain embodiments, the amount of acid used to completely or partially neutralize the base polymer can be about 0.1%, 0.25%, 0.5%, 0.75%, 1%, 1.25%, 1.5%, 1.75%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, or 20%, or intermediate values thereof.

In certain embodiments, Active Particulates can contain a plasticizer in the polymer matrix, the outer coatings (e.g., the seal coat, the functional coat layer(s), and/or the over coat), or both the polymer matrix and the outer coatings. In certain embodiments, the Active Particulates can contain a surfactant in the polymer matrix, the outer coatings, or in both the polymer matrix and the outer coatings.

In certain embodiments, Active Particulates contain an active agent (e.g., an opioid) in an amount of about 0.1% to about 95% w/w of the uncoated Active Particulates, i.e., the Active Particulates before being coated with the (optional) seal coat and/or any functional coat layer(s).

In certain embodiments, the active agent is an opioid. In certain embodiments, the opioid is oxycodone, or a pharmaceutically acceptable salt thereof. In certain embodiments, the opioid is oxycodone hydrochloride. In certain embodiments, the opioid is hydrocodone, or a pharmaceutically acceptable salt thereof. In certain embodiments, the opioid is hydrocodone bitartrate. In certain embodiments, the opioid is hydromorphone, or a pharmaceutically acceptable salt thereof. In certain embodiments, the opioid is hydromorphone hydrochloride. In certain embodiments, the opioid is oxymorphone. In certain embodiments, the opioid is codeine, or a pharmaceutically acceptable salt thereof.

In certain embodiments, the polymer matrix comprises a nonionic polymer and/or a cationic polymer. Representative cationic polymers include, but are not limited to, (meth)acrylic polymers and (meth)acrylic copolymers (e.g., copolymers of alkyl (meth)acrylates and copolymers of alkylamino(meth)acrylates); quaternary ammonium (meth)acrylic polymers.

Representative nonionic polymers in the polymer matrix include, but are not limited to, a nonionic copolymer of ethyl acrylate, methyl methacrylate, and a low content of methacrylic acid ester with quaternary ammonium groups (ammonium methacrylate copolymer, Type A, NF); and nonionic polymers such as hydroxypropylcellulose (e.g., KLUCEL®, L, J, G, M and H grades (Ashland)), hydroxypropyl methylcellulose (HPMC) (e.g., METHOCEL® E, F, J, and K (Dow Chemicals)), hydroxyethylcellulose (e.g., NATRASOL L, G, M, and H grades (Ashland)), ethylcellulose (e.g., ETHOCEL® 7FP, 10FP, 45FP, and 100FP (Dow Chemicals) and N7, N10, N14, N22, N50, and N100 grades (Ashland)), cellulose acetate butyrate (e.g., CAB-381-0.5 (Eastman)), and cellulose acetate (CA-398-3, CA-398-6, CA-398-100, and CA-398-30 (Eastman)); polyvinyl acetate polymers (e.g., polyvinyl acetate-polyvinylpyrrolidone (Kollidon SR) and polyethylene oxide polymers (e.g., Polyox® WSR coagulant, Polyox® WSR-301, Polyox® WSR-303). Exemplary polyoxyethylene oxide polymers include POLYOX™ WSR N-80, POLYOX™ WSR N-750, POLYOX™ WSR N-3000, POLYOX™ WSR-205, POLYOX™ WSR N-1105, POLYOX™ WSR N-12K, POLYOX™ WSR N-60K, POLYOX™ WSR N-301, POLYOX™ WSR Coagulant, POLYOX™ WSR N-303. The exemplary polyoxyethylene oxide polymers provide different viscosities in an aqueous solution. In certain embodiments, the exemplary polyethylene oxide has an average molecular weight of about 1,000,000 (WSR-N-12K), about 4,000,000 (WSR-301), about 5,000,000 (WSR Coagulant), or about 7,000,000 (WSR-303).

Representative pH-dependent polymers include, but are not limited to, cationic pH-dependent release polymers (e.g., base polymers that are completely or partially neutralized with an acid) that are soluble in gastric fluid at a pH below about 5, but can swell and become semipermeable at a pH above about 5. In some embodiments, the cationic pH-dependent polymer matrix comprises a partially or completely neutralized EUDRAGIT® E PO, which has a molecular weight about 47,000 and a glass transition temperature about 48° C.

The polymer matrix (i.e., the polymer matrix without the active agent embedded within) can be present in the Active Particulates in a range of about 1.0% to about 95% w/w based on the total weight of the uncoated Active Particulate, in some embodiments, from about 15% to about 90% w/w based on the total weight of the uncoated Active Particulate, and in other embodiments, from about 30% to about 75% w/w based on the total weight of the uncoated Active Particulate. In certain embodiments, the polymer matrix can be present in an amount of at least about 1%, 5%, 10%, 15%, 20%, 25%, 30%, 35%, 40%, 45%, 50%, 55%, 60%, 65%, 70%, 75%, 80%, 85%, 90%, or 95% w/w, or intermediate values thereof, based on the total weight of the uncoated Active Particulate.

In certain embodiments, a plasticizer can be added to increase the elasticity of the polymer in Active Particulates. In certain embodiments, the plasticizer makes the Active Particulate crush-resistant. In certain embodiments, the plasticizer is soluble in both aqueous and nonaqueous solvents that are commonly used to extract opioids and other abuse-prone drugs from commercial formulations. In certain embodiments, the plasticizer acts as an aversion agent. In certain embodiments, the plasticizer acts as a tissue irritant that causes discomfort if administered in conjunction with an active agent with which it is coextracted.

Representative plasticizers include, but are not limited to liquid esters, (e.g., triethyl citrate, propylene glycol, polyethylene glycols, triacetin, diethylene glycol monoethyl ether, dibutyl sebacate, and diethyl phthalate). In certain embodiments, the dielectric constant values of the plasticizer are in a range of about 5 to about 60. In other embodiments, the dielectric constant values of the plasticizer are in a range of about 10 to about 40.

In certain embodiments, the plasticizer can be present in an amount that is sufficient to make the Active Particulates substantially crush-resistant, but not in quantities that negatively impact the dissolution of the active agent when taken in a manner consistent with the manufacturer's instructions or in a manner not prescribed. In certain embodiments, the plasticizer can be present in amounts that result in discomfort to the abuser when the plasticizer is co-eluted with the active agent and administered in a manner inconsistent with the manufacturers' and/or physicians' instructions. In certain embodiments, the amount of plasticizer provides an adequate rubbery state and elongation property to the polymer to achieve crush-resistance, making it difficult to pulverize the Active Particulates into a fine powder, thereby deterring abuse.

In certain embodiments, the plasticizer can be present in a range of about 0.1% to about 30% w/w of the uncoated Active Particulates. In certain embodiments, the plasticizer can be present in a range from about 2.0% to about 15% w/w of the uncoated Active Particulates. In certain embodiments, the plasticizer can be present in an amount of about 0.2% to about 27.5%, about 0.3% to about 25%, about 0.4% to about 22.5%, about 0.5% to about 20%, about 0.6% to about 17.5%, about 0.7% to about 15%, about 0.8% to about 12.5%, about 0.9% to about 10%, about 1% to about 7.5%, or about 2.5% to about 5% w/w of the uncoated Active Particulate. In certain embodiments, the plasticizer can be present in an amount of at least about 0.1%, 0.2%, 0.5%, 1%, 5%, 10%, 15%, 20%, 25%, or 30% w/w, or intermediate values thereof, of the uncoated Active Particulate. In certain embodiments, the plasticizer can be present in an amount of about 2%, 3%, 4%, 6%, or 8% w/w, or intermediate values thereof, of the uncoated Active Particulate.

In certain embodiments, the Active Particulate matrix further comprises at least one surfactant. In certain embodiments, the pharmaceutically acceptable surfactants that are useful in the practice of the present disclosure have solubility in oils, co-solvents, or aqueous media. In certain embodiments, the surfactant component helps in modulating the solubility of the active agent. In certain embodiments, the surfactant helps to reducing the abuse potential by a dual mechanism. First, it elicits the irritant response when administered “as is” by nasal or injection routes, and second, by co-eluting with the drug when extracted with the commonly used solvents such as aqueous and organic solvents. Surfactants produce tissue irritation when applied to nasal mucosa and will cause local irritation at an injection site. Further, docusate sodium is commonly used as a stool softener/laxative, so while providing some relief for opioid-induced constipation at the intended dose, it can cause undesirable gastrointestinal effects if large quantities are ingested. Similar gastrointestinal effects can be obtained by ingesting other surfactants. In certain embodiments, the surfactant is present in an amount that results in discomfort to the abuser when the surfactant is co-eluted with the pharmaceutically active agent. The hydrophilic-lipophilic balance (“HLB”) values of the surfactants are in a range of about 4 to about 30.

Types of surfactants that can be useful in the practice of the present disclosure include nonionic surfactants (e.g., esters of fatty acids, especially of C8-C24 and preferably of C16-C22, and fatty acid esters of polyols such as glycerol or sorbitol); sorbitan fatty acid esters ethoxylated with from 2 to 30 moles of ethylene oxide; polyethylene glycol fatty acid esters; polyethyleneglycol esters and polyethyleneglycol ethers; and polyethoxylated carboxylic acids (e.g., PEG-35 castor oil, PEG-40 castor oil, steareth-2 (e.g., Brij 72, Uniqema), steareth-21 (e.g., Brij 721, Uniqema), ceteareth-25 (e.g., Cremophor A25, BASF Cooperation), PEG-7 hydrogenated castor oil (e.g., Cremophor WO7, BASF Cooperation), and PEG-30 dipolyhydroxystearate (e.g., Arlacel P 135, Uniqema)); block copolymers based on ethylene oxide and propylene oxide (e.g., PLURONIC® (e.g., 188 or 407 (BASF)); dioctyl sodium sulfosuccinate (docusate sodium); sodium lauryl sulfate; PEG-32 glyceryl laurate; PEG-32 glyceryl palmitostearate; PEG-8 glyceryl caprylate/caprate; PEG-6 glyceryl caprylate/caprate; macrogol 15 hydroxystearate; polyoxyethylene 20 sorbitan monolaurate (polysorbate 20); polyoxyethylene 20 sorbitan monooleate (polysorbate 80); sorbitan monolaurate; sorbitan monooleate; and polyoxyl 40 stearate. Anionic surfactants (e.g., alkyl ether sulfates and sulfosuccinates) can also be useful. Alternatively, cationic and amphoteric surfactants such as phospholipids, lysophospholipids, and PEGylated phospholipids can also be used. Additional useful surfactants include, vitamin E and derivatives thereof (e.g., PEGylated derivatives of vitamin E such as tocopherol PEG succinate, tocopheryl polyethylene glycol sebacate, tocopheryl polyethylene glycol dodecanodioate, tocopheryl polyethylene glycol suberate, tocopheryl polyethylene glycol azelaate, tocopheryl polyethylene glycol citraconate, tocopheryl polyethylene glycol methylcitraconate, tocopheryl polyethylene glycol itaconate, tocopheryl polyethylene glycol maleate, tocopheryl polyethylene glycol glutarate, tocopheryl polyethylene glycol glutaconate, tocopheryl polyethylene glycol fumarate, tocopheryl polyethylene glycol phthalate, tocotrienol polyethylene glycol succinate, tocotrienol polyethylene glycol sebacate, tocotrienol polyethylene glycol dodecanodioate, tocotrienol polyethylene glycol suberate, tocotrienol polyethylene glycol azelaate, tocotrienol polyethylene glycol citraconate, tocotrienol polyethylene glycol methylcitraconate, tocotrienol polyethylene glycol itaconate, tocotrienol polyethylene glycol maleate, tocotrienol polyethylene glycol glutarate, tocotrienol polyethylene glycol glutaconate, tocotrienol polyethylene glycol fumarate, and tocotrienol polyethylene glycol phthalate). See, e.g., USPAP 2014/0271593, hereby incorporated-by-reference herein.

In certain embodiments, the surfactant can be present in a range of about 0.01% to about 15% w/w of the uncoated Active Particulates. In certain embodiments, the surfactant can be present in a range from about 0.15% to about 5% w/w of the uncoated Active Particulates. In certain embodiments, the surfactant can be present in an amount of about 0.025 to about 12.5%, about 0.05% to about 10%, about 0.075% to about 7.5%, about 0.1% to about 5%, about 0.25% to about 2.5%, or about 0.5% to about 1% w/w of the uncoated Active Particulates. In certain embodiments, the surfactant can be present in an amount of about 0.2%, about 0.5%, about 2%, or about 2.2%, w/w of the uncoated Active Particulates.

In certain embodiments, certain combinations of aversion agents (e.g., plasticizer and surfactant) can be used to deter abuse. Examples of such combinations include, but are not limited to, triethyl citrate and docusate sodium (DOSS™); propylene glycol and DOSS™; polyethylene glycol (PEG-400) and DOSS™; and PEG-400 or PEG-40 hydrogenated castor oil. In certain embodiments, surfactants are used as aversion agents. Examples of such surfactants include, but are not limited to, Polyoxyl 40 hydrogenated castor oil (Cremaphor RH40), PEG 35 castor oil, and Polyoxyl 35 hydrogenated castor oil (Cremaphor EL). In certain embodiments, plasticizers are used as aversion agents. Examples of such plasticizers include, but are not limited to, PEG-3350 and PEG-6000.

In certain embodiments, the Active Particulates further contain an antioxidant. In certain embodiments, the antioxidants are present in an amount sufficient to suppress degradation of high molecular weight PEO upon hot melt extrusion (HME). Polymer degradation can result in an uncontrolled release profile, particularly when active material is embedded in a matrix of PEO; this can be another cause of oxidative degradation of pharmacologically active ingredients by, e.g., radicals. When adding an excipient, such as butylated hydroxytoluene (BHT), to attempt to stabilize high molecular weight PEO polymer, it should be taken into consideration that such an excipient should be stable at elevated temperatures, e.g., hot-melt extrusion temperatures used during manufacture of Active Particulates. Antioxidants for use in the present disclosure include, but are not limited to, ascorbic acid and its salts, tocopherols, sulfite salts such as sodium metabisulfite or sodium sulfite, sodium sulfide, butylated hydroxyanisole, butylated hydroxytoluene, ascorbyl palmitate, and propyl gallate. In certain embodiments, the antioxidant can be present in a range of about 0.01% to about 2% w/w of the uncoated Active Particulates. In certain embodiments, the antioxidant can be present in a range of about 0.025% to about 1%, about 0.05% to about 0.75%, about 0.075% to about 0.5%, or about 0.1 to about 0.75% w/w of the uncoated Active Particulates. In certain embodiments, the antioxidant can be present in about 0.2%, about 0.3%, about 0.4%, or about 0.5% w/w of the uncoated Active Particulates.

In certain embodiments, the Active Particulates can be prepared in several ways known to those in the art, including HME, film melt, granulation, melt granulation, extrusion spheronization, or rotor or roller compaction. In certain embodiments, the Active Particulates, containing PEO polymers, prepared by granulation, extrusion (e.g., HME), spheronization, rotor, or roller compaction process can require curing at a temperature above the melting point of the PEO polymers. In certain embodiments, the Active Particulates (e.g., Opioid Particulates) can be prepared by an HME process. In an HME process, a thermoplastic carrier polymer (e.g., nonionic polymer and/or cationic polymer) is combined with an active agent, a plasticizer, a surfactant, as well as any optional ingredients (e.g., an ion exchange polymer, alkaline buffering agent, and/or viscosity-building agent) to form a powdery mixture. The mixture is introduced into one or two rotating screws that convey the powder into a heated zone where shear forces compound the materials until a molten mass is achieved. Hot-melt extrusion equipment typically includes an extruder, auxiliary equipment for the extruder, downstream processing equipment, and other monitoring tools used for performance and product quality evaluation. The extruder is typically composed of a feeding hopper, barrels, single or twin screws, and the die and screw-driving unit. The auxiliary equipment for the extruder mainly includes a heating/cooling device for the barrels, a conveyer belt to cool down the product, and a solvent delivery pump. The monitoring devices on the equipment include temperature gauges, a screw-speed controller, an extrusion torque monitor and pressure gauges. In certain embodiments, different shaped dies can be used. For example, extrudates can be produced by extruding the material through round-shaped dies into cooled rolls, wherein the extruded strands are cut into short cylinders using a pelletizer.

The pelletized extruded strands are subjected to an appropriate size reduction process (or processes) using co-mill or fitz mill or micropulverizer with coolant processing aids such as dry ice or liquid nitrogen.

In certain embodiments, the sizes of Active Particulates, before or after attempted grinding, are significantly large enough to prevent the Active Particulates from being snorted. In certain embodiments, the mean size distribution of the Active Particulates can be from about 125 μm to about 1000 μm (1 mm), and in some embodiments from about 250 μm to about 750 μm (as measured by weight frequency distribution using sieving method). In certain embodiments, the mean particle size of the Active Particulates is about 400 μm to about 600 μm. In certain embodiments, the mean particle size of the Active Particulates is about 500 μm.

### 5.2.3. Seal Coat

In certain embodiments, the Active Particulates can be seal coated. The seal coat can be disposed between the inner polymer matrix core (i.e., the polymer matrix with active agent embedded within) and the at least one functional coat layer (e.g., FC 1). In certain embodiments, the seal coat is disposed between drug-layered pellets/cellets and the at least one functional coat layer (e.g., FC 1). In certain embodiments, the seal coat can be made with a nonionic water-soluble polymer. In certain embodiments, the nonionic water-soluble polymer that can be included in the seal coat is a cellulose ether polymer (e.g., a water-soluble methylcellulose and/or hydroxypropyl methylcellulose polymer). In certain embodiments, the amount of the polymer ranges from about 5% to about 100% w/w of the total weight of the composition of the seal coat (also noted within as “seal coat composition”), in some embodiments from about 30% to about 95% w/w based on the total weight of the composition of the seal coat and in some embodiments from about 50% to about 75% w/w based on the total weight of the seal coat composition. In certain embodiments, the amount of the polymer ranges from about 10% to about 95%, about 15% to about 90%, about 20% to about 85%, about 25% to about 80%, about 30% to about 75%, about 35% to about 70%, about 40% to about 65%, about 45% to about 60%, or about 50% to about 55% w/w of the total weight of the seal coat composition.

In certain embodiments, the composition of the seal coat can also include additional excipients such as an anti-tacking agent (e.g., talc, magnesium trisilicate, colloidal silicon dioxide (e.g., CAB-O-SIL®)) and a plasticizer; the plasticizer can be the same as or different from the plasticizer(s) that can be present in Active Particulates. In certain embodiments, the amount of the additional excipients, when present, can range from about 0.1% to about 40% w/w of the total weight of the seal coat composition, and in some embodiments from about 0.5% to about 10% w/w based on the total weight of the seal coat composition. In certain embodiments, the additional excipients are present at about 0.5% or about 4% w/w based on the total weight of the seal coat composition. In certain embodiments, the additional excipients are present at about 0.25% or about 35%, about 0.5% or about 30%, about 0.75% or about 25%, about 1% or about 20%, about 2.5% or about 15%, or about 5% or about 10% w/w based on the total weight of the seal coat composition.

In certain embodiments, the seal coat composition can also include an amount of the active agent, which can be therapeutically effective in and of itself, as well as the plasticizer and/or the surfactant, as well as other excipients and ingredients such as one or more solvents (both aqueous and organic, e.g., ethanol), as well as other excipients that can also be included in the seal coat composition.

In certain embodiments, the seal coat can be present in a range of about 0.1% to about 40% w/w of the uncoated Active Particulates, i.e., the Active Particulates before being coated with the (optional) seal coat, the Functional Coat(s), and the over coat. In certain embodiments, the seal coat can be present in a range from about 5% to about 25% w/w of the uncoated Active Particulates. In certain embodiments, the seal coat can be present in an amount of about 5% or about 15% w/w of the uncoated Active Particulates. In certain embodiments, the seal coat can be present in a range of about 0.2% to about 37.5%, about 0.3% to about 35%, about 0.4% to about 32.5%, about 0.5% to about 30%, about 0.6% to about 27.5%, about 0.7% to about 25%, about 0.8% to about 22.5%, about 0.9% to about 20%, about 1% to about 17.5%, about 2.5% to about 15%, about 5% to about 12.5%, or about 7.5% to about 10% w/w of the total weight of the uncoated Active Particulates. In certain embodiments, the seal coat can be present in an amount of at least about 0.1%, at least about 0.2%, at least about 0.5%, at least about 1%, at least about 5%, at least about 10%, at least about 15%, at least about 20%, at least about 25%, at least about 30%, at least about 35%, or at least about 40% w/w of uncoated Active Particulates.

### 5.2.4. Functional Coat Layers

In certain embodiments, the Active Particulates are coated with a functional coat layer(s) (e.g., FC 1, FC 0, and/or FC 2). In certain embodiments, one or more functional coat layers (e.g., FC 1, FC 0, and/or FC 2) include a water-insoluble nonionic polymer (such as a polymer that is not soluble in physiological fluids and common organic solvents such as ethanol) and a cationic polymer (e.g., a base polymer completely or partially neutralized with an acid) that is soluble in gastric fluids and behaves as a pore former at pH below about 5.

In certain embodiments, one or more functional coat layers of the Active Particulates comprise (1) at least a water-insoluble nonionic polymer, e.g., cellulose acetate, cellulose acetate-based polymers (e.g. OPADRY® CA, cellulose acetate butyrate, cellulose acetate propionate, and the like), polyvinyl acetate polymers, polyvinyl acetate-based copolymers (e.g., KOLLIDON® SR), ethylcellulose (e.g., ETHOCEL™), EUDRAGIT® RL 100, EUDRAGIT® RL PO, EUDRAGIT® RS 100, EUDRAGIT® RS PO, EUDRAGIT® NE 30 D, EUDRAGIT® NE 40 D, and the like, or a blend thereof and (2) at least a partially neutralized base polymer copolymer (e.g., dimethylaminoethyl methacrylate, butyl methacrylate, and methyl methacrylate copolymer partially neutralized with an acid, e.g., succinic acid).

In certain embodiments, one or more functional coat layers comprise at least cellulose acetate and a dimethylaminoethyl methacrylate, butyl methacrylate, and methyl methacrylate copolymer. In certain embodiments, the dimethylaminoethyl methacrylate, butyl methacrylate, and methyl methacrylate copolymer is EUDRAGIT® E PO. In certain embodiments, EUDRAGIT® E PO is a completely neutralized cationic salt. In certain embodiments, EUDRAGIT® E PO is partially neutralized, e.g., a mixture of EUDRAGIT® E PO and a cationic salt thereof. In certain embodiments, partially neutralized EUDRAGIT® E PO and cationic salt thereof can be from about 100:0.001 to about 0.001:100 wt % ratio. In certain embodiments, partially neutralized EUDRAGIT® E PO and cationic salt thereof can be from about 90:10 to about 100:0.001 wt % ratio. In certain embodiments, partially neutralized EUDRAGIT® E PO and a cationic salt thereof can be in a ratio of about 91:9, 92:8, 93:7, 94:6, 95:5, 96:4, 97:3, 97.5:2.5, 98:2, 98.5:1.5, 98.75:1.25, 99:1, or 99.5:0.5 wt %, or intermediate values thereof.

In certain embodiments, a functional coat layer comprising cellulose acetate (“CA”) (and/or CA-based polymer blends) together with the partially neutralized EUDRAGIT® E PO becomes semipermeable/less permeable at a pH greater than about 5, thereby significantly reducing drug release. In certain embodiments, the ratio of CA to partially neutralized EUDRAGIT® E PO can be from about 10:90 to about 90:10, from about 20:80 to about 80:20, from about 30:70 to about 70:30, from about 40:60 to about 60:40, or about 50:50 wt % ratio. In certain embodiments, CA and neutralized EUDRAGIT® E PO can be from about 45:55, about 50:50, about 55:45, and about 60:40 wt % ratio.

In certain embodiments, the water-insoluble nonionic polymer is a polyvinyl acetate polymer (“PVA polymer”) or a PVA-based polymer or copolymer. In certain embodiments, a functional coat layer comprising the PVA-based polymer together with the pH-dependent pore former becomes semipermeable/less permeable at pH greater than 5, thereby significantly reducing drug release. In certain embodiments, the ratio of PVA-based polymer to pore former (i.e., PVA-based polymer: pore former) can be from about 10:90 to about 90:10, from about 20:80 to about 80:20, from about 30:70 to about 70:30, from about 40:60 to about 60:40, and from about 50:50 wt % ratio. In certain embodiments, the ratio of PVA-based polymer to pore former can be from about 45:55, about 50:50, about 55:45, and about 60:40 wt % ratio.

In certain embodiments, if three or more dosage units are taken, release of the active agent from the dosage form is significantly reduced. In certain embodiments, the release is reduced by about 25%, 35%, 45%, 55%, 65%, 75%, 85%, 95%, 96%, 97%, 98%, 99%, or increments therein. In certain embodiments, the release is reduced from about 30% to about 90%, about 40% to about 80%, or about 50% to about 70%.

In certain embodiments, the composition of the functional coating can also include an anti-tacking agent (e.g., talc, magnesium trisilicate, colloidal silicon dioxide (e.g.,)) CAB-O-SIL® and/or a plasticizer.

In certain embodiments, the functional coating prevents the extraction of the active agent in water and in water/alcohol mixtures.

In certain embodiments, FC 1 can be present in a range of about 1% to about 100% w/w of the uncoated or seal coated Active Particulates (e.g., the polymer matrix with active agent embedded within, also including the optional seal coat, if present). In certain embodiments, the FC 1 can be present in a range of about 10% to about 90%, about 15% to about 80%, about 20% to about 70%, about 25% to about 60%, about 30% to about 55%, or about 35% to about 50% w/w of the uncoated or seal coated Active Particulates. In certain embodiments, FC 1 can be present in a range of about 100%, 95%, 90%, 85%, 80%, 75%, 70%, 65%, 60%, 55%, 50%, 45%, 40%, 35%, 30%, 25%, 20%, 15%, 10%, 5%, or 1% w/w of the uncoated or seal coated Active Particulates. In certain embodiments, FC 1 can be present in about 100% w/w of the uncoated or seal coated Active Particulates. In certain embodiments, FC 1 can be present in about 60% w/w of the uncoated or seal coated Active Particulates.

In certain embodiments, the Active Particulates also can be coated with additional functional coat layers (e.g., FC 2 and/or FC 0) to further enhance ODP features. In certain embodiments, the FC 1-coated Active Particulates can be further coated with an additional functional coat layer FC 2. In certain embodiments, the FC 1 and FC 2 are present in a ratio of about 100:0 to 0:100. In certain embodiments, FC 1 and FC 2 are present in a ratio of about 90:10, 80:20, 70:30, 60:40, 50:50, 40:60, 30:70, 20:80, and 10:90. In certain embodiments, FC 2 and/or FC 0 can comprise a cationic polymer. In certain embodiments, the cationic polymer is a partially neutralized free base, e.g., as a mixture of free base form and a cationic salt thereof. In certain embodiments, the cationic polymer is present in free base form. In certain embodiments, the cationic polymer is a completely neutralized free base. In certain embodiments, FC 2 and/or FC 0 can comprise a cationic polymer (in free base form and/or a cationic salt thereof) and a water-insoluble nonionic polymer.

In certain embodiments, the composition of the FC 2 and/or FC 0 can also include an anti-tacking agent (e.g., talc, magnesium trisilicate, colloidal silicon dioxide (e.g., CAB-O-SIL®)) and/or a plasticizer.

In certain embodiments, Active Particulates can comprise one, two, or three functional coat layers (e.g., FC 1, or FC 1 and FC 0 and/or FC 2). In certain embodiments, Active Particulates can comprise more than three functional coat layers (e.g., four or five functional coat layers). In certain embodiments, any one or more of the functional coat layers can comprise a cationic polymer in the absence of a water-insoluble nonionic polymer. In certain embodiments, any one or more of the functional coats can comprise a cationic polymer in the presence of a water-insoluble nonionic polymer; in such embodiments, the ratio of water-insoluble nonionic polymer to cationic polymer can be from about 90:10 to about 10:90.

### 5.2.5. Over Coat

In certain embodiments, the functional coated Active Particulates (i.e., with or without FC 2) include an over coat to prevent/minimize the interaction of EUDRAGIT® E PO (e.g., in FC 1 and/or FC 2) with the alkaline agent present in the Triggering Particulates. The over coat can include a nonionic polymer (e.g., hydroxypropyl methylcellulose).

In certain embodiments, the composition of the over coat can also include additional excipients such as an anti-tacking agent (e.g., talc, magnesium trisilicate, colloidal silicon dioxide (e.g., CAB-O-SIL®)) and a plasticizer; the plasticizer can be the same as or different from the plasticizer(s) that can be present in Active Particulates.

In certain embodiments, the over coat can be present in a range of about 5% to about 50% w/w of the functional coated Active Particulates (i.e., the polymer matrix with active agent embedded within, (optional) seal coat, and one or more functional coat layers). In certain embodiments, the over coat can be present in a range of about 10% to about 50%, about 10% to about 45%, about 10% to about 35%, about 10% to about 30%, about 15% to about 40%, about 15% to about 25%, about 20% to about 35%, or about 25% to about 30% w/w of the functional coated Active Particulates.

### 5.2.6. Crush and Extractability Resistance

In certain embodiments, the Active Particulates are at least partially crush-resistant, nongrindable, and nonextractable. In certain embodiments, they are substantially noncrushable, nongrindable, and nonextractable, thereby making the active agent difficult to abuse. For example, the Active Particulates resist abuse via, but not limited to, crushing and swallowing; crushing and insufflating/inhaling nasally (“snorting”); crushing and smoking; or crushing, dissolving, and injecting (subcutaneously (i.e., skin popping), intravenously, or intramuscularly). In certain embodiments, the Active Particulates cannot be ground or crushed into particles small enough to be effectively snorted or injected. In certain embodiments, the Active Particulates cannot be pulverized into fine powder by mechanical grinding.

The crush-resistance of the Active Particulates can be determined by measurement of the crushing strength required to deform the Active Particulates without any evidence of fragmentation or breaking into smaller pieces or powder using an Instron Tester or equivalent. In some embodiments, the Active Particulates of the disclosure can withstand a crushing strength ranging from 300-1000 N. Abuse deterrence can be tested by examining the mean particle size following the physical and/or mechanical manipulation, with or without thermal pretreatment, of the Active Particulate. For example, the Active Particulates can be subjected to grinding/crushing in a coffee grinder, mill, mortar and pestle, a food processor, a blender, etc. For example, Active Particulates can be placed in a coffee grinder (e.g., Hamilton Beach Coffee Grinder) and ground for several cycles (e.g., at a 10-cup setting for 8 cycles of 30 seconds each).

The mean particle size of the particulates after grinding can be measured using sieve analysis that gathers particulates of the same size into groups based on particle size. The weight of the particulates in each group can be measured and compared to an unground sample.

In certain embodiments, the mean particle size after grinding the Active Particulates is about 500 μm (with a range of about 250 μm to about 1000 μm), as measured by weight frequency distribution using sieving method. In certain embodiments, the mean particle size after grinding the Active particulates is greater than about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 525 μm, about 550 μm, about 575 μm, about 600 μm, about 625 μm, about 650 μm, about 675 μm, or about 700 μm.

Abuse deterrence can be tested by examining the syringeability of the Active Particulates either before or after grinding. For example, syringeability can be tested by examining the difficulty of drawing a solution of the dosage form comprising Active Particulates, dissolved in varying types of solvents (e.g., water) and volumes of solvent (e.g., 2-10 ml) through, e.g., an 18-gauge syringe needle. The syringeability can also be tested by determining the amount of active ingredient present in the withdrawn liquid.

Abuse deterrence can also be tested by examining the extractability of active agent from the Active Particulates before and after grinding.

### 5.3. Triggering Particulates

In certain embodiments, Triggering Particulates (e.g., Triggering Granules) can contain a combination of at least one alkaline agent (e.g., magnesium hydroxide (increases pH from 1.6 to greater than 5.0)) and/or at least one pH-stabilizing agent (e.g., di- and/or tricalcium phosphate (maintains the elevated pH of greater than 5.0 for up to about 30 minutes, about one hour, or about two hours)). In certain embodiments, ingestion of one or two dosage units (i.e., one or two tablets or capsules) results in little or no increase in pH of the gastric fluids. In certain embodiments, ingestion of multiple dosage units (e.g., three or more) results in the alkaline agent increasing the pH very rapidly above about 5. In certain embodiments, the pH-stabilizing agent acts to maintain or stabilize the increased pH caused by the alkaline agent. For example, ingestion of multiple dosage units results in (a) a rapid increase in pH caused by the alkaline agent; (b) modulation of pore formation in the functional coat; and (c) a decrease in the rate of release of the active agent (e.g., an opioid) from the Active Particulate. In certain embodiments, upon ingestion of multiple dosage units (e.g., three or more), the pH of the gastric fluid increases very rapidly above a pH of about 5 (e.g., in about one to about five minutes). In certain embodiments, the increase in the pH of the gastric fluid upon taking multiple dosage units occurs in about two to about three minutes.

In certain embodiments, the alkaline agent for use in the Triggering Particulates include, but are not limited to, aluminum hydroxide, sodium hydroxide, potassium hydroxide, calcium hydroxide, magnesium hydroxide, calcium carbonate, sodium carbonate, potassium bicarbonate, sodium bicarbonate, sodium oxide, calcium oxide, magnesium oxide, aluminum oxide, potassium oxide, ammonia, tertiary sodium phosphate, diethanolamine, ethylenediamine, N-methylglucamine, L-lysine, and combinations thereof. In certain embodiments, the alkaline agent is magnesium hydroxide.

In certain embodiments, the alkaline agent is present in an amount that when a single dosage unit is taken, it does not alter the pH of the gastric fluid. In certain embodiments, the alkaline agent is present in an amount from about 30% to about 90% w/w of total Triggering Particulates. In certain embodiments, the alkaline agent is present in an amount from about 35% to about 85%, about 40% to about 80%, about 45% to about 75%, about 50% to about 70%, or about 55% to about 65% w/w of total Triggering Particulate. In certain embodiments, the alkaline agent is present in an amount from about 40% to about 90%, about 50% to about 80%, or about 60% to about 70%, w/w of the total Triggering Particulate. In certain embodiments, the alkaline agent is present in an amount from about 80% to about 85% w/w of the total Triggering Particulate. In certain embodiments, the alkaline agent is present in an amount from about 10% to about 60%, about 20% to about 50%, or about 30% to about 40% w/w of the total weight of the dosage form. In certain embodiments, the alkaline agent is present in an amount from about 15% to about 55%, about 25% to about 45%, or about 27% to about 39% w/w of the total weight of the dosage form. In certain embodiments, the alkaline agent is present in an amount of about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45% w/w of the total weight of the dosage form, or increments therein.

In certain embodiments, the pH-stabilizing agents for use in the Triggering Particulates include, but are not limited to, bismuth aluminate, bismuth carbonate, bismuth subcarbonate, bismuth subgallate, bismuth subnitrate, calcium phosphate, dibasic calcium phosphate, dihydroxyaluminum aminoacetate, dihydroxyaluminum glycine, magnesium glycinate, sodium potassium tartrate, tribasic sodium phosphate, tricalcium phosphate, and combinations thereof. In certain embodiments, the pH-stabilizing agent is a combination of dibasic calcium phosphate/tricalcium phosphate. In certain embodiments, the ratio of dibasic calcium phosphate to tricalcium phosphate (i.e., dibasic calcium phosphate: tricalcium phosphate) is about 1:1 to about 1:5 wt % ratio. In certain embodiments, the ratio of dibasic calcium phosphate to tricalcium phosphate is about 1:1.25 to about 1:4.75, about 1:1.5 to about 1:4.5, about 1:1.75 to about 1:4.25, about 1:2 to about 1:4, about 1:2.25 to about 1:3.75, about 1:2.5 to about 1:3.5, or about 1:2.75 to about 1:3.25 wt %. In certain embodiments, the pH-stabilizing agent is anhydrous dibasic calcium phosphate.

In certain embodiments, the pH-stabilizing agent is present in an amount that when a single dosage unit is taken, it does not alter the pH of the gastric fluid, but when multiple dosage units are taken (e.g., three or more dosage units), the pH-stabilizing agent maintains the elevated pH levels caused by the alkaline agent. In certain embodiments, the pH-stabilizing agent is present in an amount sufficient to maintain or stabilize the pH of the gastric fluid above about 5.0 for up to five hours. In certain embodiments, the pH-stabilizing agent is present in an amount sufficient to maintain the pH of the gastric fluid above about 5.0 for about one to about two hours. In certain embodiments, the pH-stabilizing agent is present in an amount sufficient to maintain the pH of the gastric fluid above about 5.0 for at least about 1 hour, at least about 1.25 hours, at least about 1.5 hours, at least about 1.75 hours, at least about 2 hours, at least about 2.25 hours, at least about 2.5 hours, at least about 2.75 hours, at least about 3 hours, at least about 3.25 hours, at least about 3.5 hours, at least about 3.75 hours, at least about 4 hours, at least about 4.25 hours, at least about 4.5 hours, at least about 4.75 hours, at least about 5 hours.

In certain embodiments, the pH-stabilizing agent is present in an amount from about 10% to about 60% w/w of total Triggering Particulates. In certain embodiments, the pH-stabilizing agent is present in an amount from about 12.5% to about 57.5%, about 15% to about 55%, about 17.5% to about 52.5%, about 20% to about 50%, about 22.5% to about 47.5%, about 25% to about 45%, about 27.5% to about 42.5%, about 30% to about 40%, or about 32.5% to about 37.5% w/w of total Triggering Particulates. In certain embodiments, the pH-stabilizing agent is present in an amount from about 15% to about 40%, or about 20% or about 30% w/w of total Triggering Particulates.

In certain embodiments, the alkaline agent and the pH-stabilizing agent (combined) (e.g., included in the Triggering Particulates) are present in an amount of less than 60% w/w (i.e., 60 wt %) of the total dosage form (or pharmaceutical composition). In certain embodiments, the alkaline agent and the pH-stabilizing agent (combined) are present in an amount of less than 60%, less than 55%, less than 50%, less than 45%, less than 44%, less than 43%, less than 42%, less than 41%, less than 40%, less than 39%, less than 38%, less than 37%, less than 36%, less than 35%, less than 34%, less than 33%, less than 32%, less than 31%, less than 30%, less than 29%, less than 28%, less than 27%, less than 26%, less than 25%, less than 24%, less than 23%, less than 22%, less than 21%, less than 20%, less than 19%, less than 18%, less than 17%, less than 16%, or less than 15%, w/w of the total dosage form.

In certain embodiments, the Triggering Particulates include a binder, a disintegrant, filler (or diluents), and/or a lubricant.

Binders according to the present disclosure include, but are not limited to, hydroxypropyl celluloses in various grades, hydroxypropyl methylcelluloses in various grades, polyvinylpyrrolidones in various grades, copovidones, powdered acacia, gelatin, guar gum, carbomers, methylcelluloses, polymethacrylates, and starches.

Disintegrants according to the present disclosure include, but are not limited to, carmellose calcium, carboxymethylstarch sodium, croscarmellose sodium, crospovidone (POLYPLASDONE™— crosslinked homopolymer of N-vinyl-2-pyrrolidone), low-substituted hydroxypropyl celluloses, sodium starch glycolate, colloidal silicon dioxide, alginic acid and alginates, acrylic acid derivatives, and various starches.

Lubricants according to the present disclosure include, but are not limited to, magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and any combinations thereof.

The Triggering Particulates can be prepared by any granulation method known to those of skill in the art. For example, the Triggering Particulates can be made by dry granulation (e.g., direct blend, compacting and densifying the powders), wet granulation (e.g., addition of a granulation liquid onto a powder bed under the influence of an impeller or air), or hot melt extrusion (HME). In certain embodiments, Triggering Particulates are made by wet granulation. In certain embodiments, Triggering Particulates are made by HME. The granulation product obtained can be milled to achieve uniform granules. The granules obtained can be subsequently coated with an aqueous dispersion.

In certain embodiments, the mean particle size distribution of the Triggering Particulates is about 100 μm to about 1000 μm. In certain embodiments, the mean particle size distribution of the Triggering Particulates is about 150 μm to about 950 μm, about 200 μm to about 900 μm, about 250 μm to about 850 μm, about 300 μm to about 800 μm, about 350 μm to about 750 μm, about 400 μm to about 700 μm, about 450 μm to about 650 μm, or about 500 μm to about 600 μm. In certain embodiments, the mean particle size distribution of Triggering Particulates is about 300 μm to about 800 μm.

### 5.4. Viscosity Enhancing Particulates

In certain embodiments, Viscosity Enhancing Particulates (e.g., Viscosity Enhancing Granules) increase the viscosity of the dosage form when added to a dissolution medium (e.g., water), thus impeding the ability to extract the active agent from the dosage form, or to pass the dissolution medium with the active agent through a needle for injection purposes.

In certain embodiments, the increase in viscosity can also reduce the potential absorption of the active agent when taken in amounts in excess of two dosage units (e.g., three or more dosage units). As the viscosity of the solution in the GI tract increases, the active agent is eventually entrapped in a polymer gel matrix and the dosage form is transformed from an immediate release formulation to the equivalent of an extended release formulation. It is believed that the ingestion of increasing quantities of the formulation will not proportionally increase the maximum concentration (Cₘₐₓ) to reach the full potential of abusive effects (e.g., euphoria, sedation, and/or relaxation) of the active agent. In addition, it will take a longer time to reach maximum concentration (Tₘₐₓ). The result will be a reduced desirability of deliberately abusing or overdosing on the active agent.

In certain embodiments, the Viscosity Enhancing Particulates contain a viscosity-building polymer. In certain embodiments, the viscosity-building polymer is present in an amount that is sufficient to increase the viscosity of the proximal fluid in the GI tract if multiple doses, e.g., three or more dosage units, are taken, e.g., deliberately for the purpose of abuse. In certain embodiments, the viscosity-building polymer is present in an amount that prevents syringeability by rapidly forming a gelatinous mass that resists passage through a needle when one or more units are subjected to incubation in about 10 ml of aqueous or nonaqueous media.

In certain embodiments, the Viscosity Enhancing Particulates include a polymer matrix that can include a nonionic polymer (e.g., polyethylene oxide (PEO) polymers such as Polyox® WSR coagulant, Polyox® WSR-301, Polyox® WSR-303) and/or pH-dependent polymers (e.g., carbomers such as Carbopol 934P, Carbopol 971P, Carbopol 974P).

In certain embodiments, Viscosity Enhancing Particulates include an antioxidant, a plasticizer, and/or a surfactant, each of which can be the same or different from those used in the Active Particulates. In certain embodiments, the Viscosity Enhancing Particulates matrix further includes a glidant (e.g., talc, colloidal silicon dioxide, magnesium trisilicate, powdered cellulose, starch, and tribasic calcium phosphate). In certain embodiments, the Viscosity Enhancing Particulates matrix further includes a disintegrant, which can be the same or different from those used in the Triggering Particulates.

In certain embodiments, the viscosity-building polymer is present in an amount that does not retard the release of the active agent from a single dose administration, but does slow down the release of the active agent when multiple dosage units are taken together (e.g., three or more dosage units). In certain embodiments, the viscosity-building polymer is present in an amount from about 2% to about 60% w/w of total Viscosity Enhancing Particulates. In certain embodiments, the viscosity-building polymer is present in an amount from about 5% to about 55%, about 10% to about 50%, about 15% to about 45%, about 20% to about 40%, or about 25% to about 35% w/w of total Viscosity Enhancing Particulates. In certain embodiments, the viscosity-building polymer is present in an amount from about 10% to about 50%, or about 15% to about 20%, w/w of total Viscosity Enhancing Particulates.

Viscosity Enhancing Particulates can be prepared by any granulation method known to those of skill in the art. For example, the Viscosity Enhancing Particulates can be made by dry granulation (e.g., direct blend, compacting and densifying the powders), wet granulation (e.g., addition of a granulation liquid onto a powder bed under the influence of an impeller or air), melt granulation, hot-melt extrusion, extrusion spheronization, or rotor granulation. The granulation product obtained can be milled to achieve uniform granules. The granules obtained can be subsequently coated with an aqueous dispersion.

In certain embodiments, the mean particle size distribution of the Viscosity Enhancing Particulates is about 125 μm to about 1000 μm. In certain embodiments, the mean particle size distribution of the Viscosity Enhancing Particulates is about 150 μm to about 950 μm, about 200 μm to about 900 μm, about 250 μm to about 850 μm, about 300 μm to about 800 μm, about 350 μm to about 750 μm, about 400 μm to about 700 μm, about 450 μm to about 650 μm, or about 500 μm to about 600 μm. In certain embodiments, the mean particle size distribution of Viscosity Enhancing Particulates is about 250 μm to about 750 μm.

### 5.5. Particulate and Multi-Particulate Dosage Forms

The present disclosure combines ADF and ODP properties in a single solid oral immediate release dosage form and thus addresses multiple health-related concerns, especially regarding habit-forming active agents for which there is a high propensity for abuse (e.g., opioids). In certain embodiments, the abuse deterrence and/or overdose protection activates after the ingestion of three or more dosage units (e.g., three or more tablets/capsules). In certain embodiments, the abuse deterrence and/or overdose protection activates when the multiple dosage units are taken at once. In certain embodiments, the abuse deterrence and overdose protection can activate when the multiple dosage units are taken in tandem. In certain embodiments, release of the active agent after ingesting one to two dosage units results in the dosage form maintaining its (their) immediate release properties (i.e., there is no (or minimal) effect on the release of the active agent from the dosage form(s)) in fed and fasted state. In certain embodiments, one or more functional coat layers, e.g., FC 0, FC1, and FC2, in Opioid Particulates contain partially neutralized EUDRAGIT® E PO. In certain embodiments, EUDRAGIT® E PO is partially neutralized with an acid, e.g., neutralized as a cationic salt with succinic acid. In certain embodiments, succinic acid can be about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, or 2.5% w/w, or intermediate values thereof, of the dosage form. In certain embodiments, succinic acid can be about 0.25 to about 5% w/w of the dosage form. In certain embodiments, succinic acid can be about 1, 2, 3, 4, 5, 6, 7, 8, 9, 10, 11, 12, 13, 14, 15, 16, 17, 18, 19, or 20% w/w, or intermediate values thereof, of the dosage form. In certain embodiments, the cationic salt of EUDRAGIT® E PO present in the functional coat maintains immediate release properties of the dosage form, independent of fed or fasted condition. In certain embodiments, if three or more dosage units are taken, the pH of the gastric fluid increases to greater than about 5, and the release of the active agent from the dosage form is significantly reduced. In certain embodiments, the release is reduced by more than about 25%, 30%, 35%, 40%, 45%, 50%, 55%, 60%, 65%, 70%, 75%, 80%, 85%, 90%, 95%, 96%, 97%, 98%, 99%, or increments therein. These dosage forms, however, are not intended to be used as an extended release or sustained release dosage form.

In certain embodiments, the presence of partially neutralized EUDRAGIT® E PO, and about 15-45% w/w magnesium hydroxide in the dosage form maintains immediate release properties of the dosage form, independent of fed or fasted state of the individual, when one or two dosage units are consumed as prescribed, while providing overdose protection when three or more dosage units are consumed together.

In certain embodiments, the pharmaceutical dosage forms contain at least one population of Active Particulates in combination with at least one population of Triggering Particulates. In certain embodiments, the alkaline agent of the Triggering Particulates increases the pH of the aqueous or nonaqueous solution to above about pH 5.0 in the presence of three or more dosage units, and the pH-stabilizing agent of the Triggering Particulates maintains the increased pH above about 5.0 for up to two hours. In certain embodiments, the functional coating of the Active Particulates only allows the release of the active agent in an aqueous or nonaqueous environment with a pH below about 5.0 and prevents or slows the release of the active agent at a pH above about 5.0. In certain embodiments, the pharmaceutical dosage forms contain at least one population of Viscosity Enhancing Particulates. In certain embodiments, the pharmaceutical dosage forms contain at least one population of Active Particulates in combination with at least one population of Triggering Particulates and at least one population of Viscosity Enhancing Particulates. In certain embodiments, the Viscosity Enhancing Particulates are present in an amount of from about 2% to about 50% of the total weight of the dosage form.

In certain embodiments, the pharmaceutical dosage forms can contain at least one population of pH-dependent Viscosity Modifying Particulates. In certain embodiments, pH-dependent Viscosity Modifying Particulates (e.g., pH-dependent Viscosity Modifying Granules) comprise pH-dependent viscosity building polymers (e.g., carbomers such as Carbopol 934P, Carbopol 971P, and Carbopol 974P). In certain embodiments, the pH-dependent viscosity building polymer can be present in an amount that does not retard the release of the active agent from a single dose administration, but does slow down the release of the active agent after multiple dosage units are taken. In certain embodiments, the pH-dependent Viscosity Modifying Particulates can be present in an amount from about 0.5% to about 15% w/w of the total weight of the dosage form. In certain embodiments, the pH-dependent Viscosity Modifying Particulates can be present in an amount from about 0.75% to about 12.5%, about 1% to about 10%, or about 2.5% to about 7.5% w/w of the total weight of the dosage form.

In certain embodiments, the pharmaceutical dosage forms can contain at least one population of Ion Exchange Resin Particulates (e.g., Amberlite™ IRP 64, Amberlite™ IRP 69). The ion exchange resins of the Ion Exchange Resin Particulates form a matrix or complex with the drug, and thus can alter the release of drug. In certain embodiments, the ion exchange resin can be present in an amount that binds to the active agent if the dosage form is tampered with, thereby preventing the release of the active agent from the dosage form. In certain embodiments, the Ion Exchange Resin Particulates can be present in a concentration of about 1-5 M and in some embodiments from about 1-3 M, based on the total molarity of the drug susceptible to abuse.

In certain embodiments, the pharmaceutical dosage forms contain at least one population of Ion Exchange Resin Particulates. In certain embodiments, the pharmaceutical dosage forms contain at least one population of Active Particulates in combination with at least one population of Triggering Particulates and at least one population of Ion Exchange Resin Particulates. In certain embodiments, the pharmaceutical dosage forms contain at least one population of Active Particulates in combination with at least one population of Triggering Particulates, at least one population of Viscosity Enhancing Particulates, and at least one population of Ion Exchange Resin Particulates. In certain embodiments, the pharmaceutical dosage forms contain at least one population of Active Particulates in combination with at least one population of Triggering Particulates, at least one population of Viscosity Enhancing Particulates, at least one population of pH-Dependent Viscosity Modifying Particulates, and at least one population of Ion Exchange Resin Particulates.

In certain embodiments, the pharmaceutical dosage forms contain at least one population of Active Particulates and Triggering Particulates.

In certain embodiments, the AD and ODP characteristics of the dosage form have synergistic effects. In certain embodiments, ODP elements of the dosage form further enhance AD features of the dosage form, i.e., in a synergistic manner. In certain embodiments, AD elements of the dosage form further enhance ODP features of the dosage form, i.e., in a synergistic manner. In certain embodiments, the ODP elements, e.g., acid labile coat (functional coat) on the Active Particulates, and/or the presence of alkaline agent in, e.g., Triggering Particulates, enhance the AD features (e.g., reduce the amount of active in the syringeable liquid by further controlling the release of the active agent from the dosage form in certain embodiments of deliberate abuse).

In certain embodiments, the pharmaceutical dosage form of the disclosure is a solid immediate release multi-particulate dosage form with abuse deterrent properties and overdose protection elements, comprising a first population of particulates comprising a therapeutically effective amount of at least one opioid embedded in a polymer matrix, and an acid labile coat, and a second population of particulates comprising an alkaline agent, wherein the abuse deterrent properties comprise resistance to extractability, and resistance to syringeability of the opioid; and the ODP elements comprise the acid labile coat, and an alkaline agent; wherein the presence of ODP elements enhance the AD properties of the dosage form in a synergistic manner. In certain embodiments, the presence of the alkaline agent reduces the amount of active agent present in a syringeable liquid to less than about 10-20%, compared with about 40% of the opioid in a dosage form without an alkaline agent. In certain embodiments, the syringeable liquid is obtained by adding at least one crushed dosage form, with or without an alkaline agent, to water at room temperature and maintaining the resulting suspension at room temperature for, e.g., 30 minutes. In certain embodiments, the dosage form without an alkaline agent comprises a single population of particulates comprising a therapeutically effective amount of at least one opioid embedded in a polymer matrix, and an acid labile coat. In certain embodiments, the dosage form without an alkaline agent comprises a tablet dosage form without Triggering Particulates.

In certain embodiments, the pharmaceutical dosage form of the disclosure is a solid immediate release multi-particulate dosage form with AD properties and an ODP element, comprising a population of particulates comprising a therapeutically effective amount of at least one opioid embedded in a polymer matrix, and an acid labile coat; wherein the AD properties comprise resistance to extractability, and resistance to syringeability of the opioid; and the ODP element comprises the acid labile coat; wherein the presence of the ODP element enhances the AD properties of the dosage form in a synergistic manner. In certain embodiments, the syringeable liquid is obtained by adding at least one crushed dosage form, with or without an alkaline agent, to water at room temperature and maintaining the resulting suspension at room temperature for, e.g., five minutes. In certain embodiments, the dosage form without an acid labile coat comprises a population of particulates comprising a therapeutically effective amount of at least one opioid embedded in a polymer matrix. In certain embodiments, the dosage form without an acid labile coat comprises a tablet dosage form without an acid labile coating on the Active Particulates.

In certain embodiments, the alkaline agent present in Triggering Particulates increases the viscosity of the dosage form by activating pH-dependent anionic polymer(s), e.g., gelling polymers such as carbomers, thereby enhancing the AD features (AD properties), such as reduced dissolution and syringeability of the dosage form, in a synergistic manner. In certain embodiments, the gelling effect of, e.g., carbomers is greatly enhanced in the raised pH resulting from the alkaline agent released from the Triggering Particulates involved in ODP. The increased AD effects of such gelling can be part of, e.g., decreases in attempted extraction, and decreased release of active agent in the stomach when three or more dosage units are ingested.

In certain embodiments, the plurality of particulate populations can be blended with other excipients and additives and compressed into a tablet or loaded into a capsule. In certain embodiments, the tablet/capsule dosage form disintegrates rapidly once in contact with aqueous medium. In certain embodiments, the capsule can be a soft or hard gelatin capsule. In certain embodiments, the capsule itself does not alter the release of the active agent.

In certain embodiments, Active Particulates are present in an amount from about 10% to about 80% w/w of the total weight of the dosage form. In certain embodiments, the Active Particulates are present in an amount from about 15% to about 75%, about 20% to about 70%, about 25% to about 65%, about 30% to about 60%, about 35% to about 55%, or about 40% to about 50% w/w of the total weight of the dosage form. In certain embodiments, the Active Particulates are present in an amount from about 50% to about 80%, about 60% to about 80%, or about 70% to about 80% w/w of the total weight of the dosage form. In certain embodiments, the Active Particulates are present in an amount from about 10% to about 70%, about 20% to about 70%, about 30% to about 70%, or about 40% to about 70% w/w of the total weight of the dosage form. In certain embodiments, the Active Particulates are present in an amount of at least about 10%, 15%, 20%, 25%, 30%, 35%, 40%, 45%, 50%, 55%, 60%, 65%, 70%, 75%, or 80% w/w of the total weight of the dosage form.

In certain embodiments, the Triggering Particulates are present in an amount from about 10% to about 50% w/w of the total weight of the dosage form. In certain embodiments, the Triggering Particulates are present in an amount from about 20% to about 42% w/w of the total weight of the dosage form. In certain embodiments, the Triggering Particulates are present in an amount from about 22% to about 40%, about 24% to about 38%, about 26% to about 36%, about 28% to about 34%, or about 30% to about 32% w/w of the total weight of the dosage form. In certain embodiments, the Triggering Particulates are present in an amount from about 20% to about 42%, about 22% to about 42%, about 24% to about 42%, about 26% to about 42%, about 28% to about 42%, about 30% to about 42%, about 32% to about 42%, about 34% to about 42%, about 36% to about 42%, about 38% to about 42%, or about 40% to about 42% w/w of the total weight of the dosage form. In certain embodiments, the Triggering Particulates are present in an amount of at least about 20%, 22%, 24%, 26%, 28%, 30%, 32%, 34%, 36%, 38%, 40%, or 42% w/w of the total weight of the dosage form.

In certain embodiments, the Viscosity Enhancing Particulates are present in an amount from about 2% to about 50% w/w of the total weight of the dosage form. In certain embodiments, the Viscosity Enhancing Particulates are present in an amount from about 5% to about 45%, about 10% to about 40%, about 15% to about 35%, or about 20% to about 30% w/w of the total weight of the dosage form.

In certain embodiments, the pH-Dependent Viscosity Modifying Particulates are present in an amount from about 0.5% to about 15% w/w of the total weight of the dosage form. In certain embodiments, the pH-Dependent Viscosity Modifying Particulates are present in an amount from about 0.75% to about 12.5%, about 1% to about 10%, or about 2.5% to about 7.5% w/w of the total weight of the dosage form.

In certain embodiments, the Ion Exchange Resin Particulates are present in a concentration of about 1-5 M, or about 1-3 M, based on the total molarity of the drug susceptible to abuse.

In certain embodiments, a single particulate population (e.g., a population of Opioid Particulates) can be blended with other excipients and additives and compressed into various tablet dosage forms, e.g., tablet, mini-tablet, tablet-in-tablet, bilayer tablet, or multilayer tablet, or loaded into a capsule, or the like. In certain embodiments, additional solid IR dosage forms, including additional particulate, tablet, and/or capsule coating regimens, are contemplated. A nonlimiting set of exemplary dosage forms follows.

In certain embodiments, the formulation is a single particulate dosage form comprising a single population of particulates (e.g., comprising a functional coat) containing at least one opioid, the particulates being compressed into a tablet/mini-tablet or filled in a capsule, and at least one alkalinizing coat covering the tablet/mini-tablet and/or capsule.

In certain embodiments, the multi-particulate dosage form is a two-particulate dosage form comprising a first population of Active Particulates containing an opioid, and a second population of Triggering Particulates, the two particulate populations being compressed into a tablet/mini-tablet or filled in a capsule.

In certain embodiments, the tablet/mini-tablet is further coated with an acid labile coat and, optionally, an alkalinizing coat on top of the acid labile coat.

In certain embodiments, Active Particulates contain an alkaline agent and, optionally, a pH-stabilizing agent in the polymer matrix.

In certain embodiments, the size of Active Particulates is, e.g., about 400 micrometers to about 2-3 mm, to provide enhanced control of release of active agent (e.g., opioid) in an ODP setting, while providing required and desired immediate release (independent of any food effect) when one or two dosage units are consumed.

In certain embodiments, the Active Particulates can have various functional coat layer(s) (e.g., without limitation, FC 0, FC 1, or FC 2, or combinations thereof).

In certain embodiments, the Active Particulates have a seal coat (optional) on top of the polymer matrix.

In certain embodiments, the Active Particulates have an over coat on top of the functional coat layer(s).

In certain embodiments, capsules contain coated Active Particulates (e.g., Opioid Particulates) coated with a functional coat layer(s) and an over coat, and Triggering Particulates.

In certain embodiments, capsules contain Triggering Particulates, and tablets/mini-tablets made from coated Active Particulates.

In certain embodiments, capsules contain tablets/mini-tablets of coated Active Particulates, and tablets/mini-tablets of Triggering Particulates.

In certain embodiments, capsules contain coated Active Particulates, and tablets/mini-tablets of Triggering Particulates.

In certain embodiments, capsules contain (1) mini-tablets/tablets comprising coated Active Particulates, and at least a portion of Triggering Particulates; and (2) a remaining portion of Triggering Particulates.

In certain embodiments, the dosage form is a bilayer tablet comprising a first layer comprising coated Active Particulates, and a second layer comprising Triggering Particulates, and the two layers are compressed into a bilayer tablet. In certain embodiments, the first layer is coated with at least one functional coat layer and an over coat on top of the at least one functional coat layer.

In certain embodiments, the dosage form is a bilayer tablet comprising a first layer comprising a coated tablet comprising Active Particulates, and a second layer comprising Triggering Particulates, and the two layers are compressed into a bilayer tablet.

In certain embodiments, the dosage form is a tablet-in-tablet dosage form comprising an inner tablet comprising coated Active Particulates, and an outer tablet, comprising Triggering Particulates, encasing the inner tablet.

In certain embodiments, the dosage form is a tablet-in-tablet dosage form comprising an inner coated tablet comprising Active Particulates, and an outer tablet, partially or completely encasing the inner tablet, comprising Triggering Particulates.

In certain embodiments, the dosage form is a capsule dosage form comprising Triggering Particulates, and compressed tablets/mini-tablets comprising Active Particulates (e.g., Opioid Particulates).

In certain embodiments, the dosage form is a capsule dosage form comprising Active Particulates (e.g., Opioid Particulates), and compressed tablets/mini-tablets comprising Triggering Particulates.

In certain embodiments, the dosage form is a capsule dosage form comprising compressed tablets/mini-tablets comprising Active Particulates (e.g., Opioid Particulates), and compressed tablets/mini-tablets comprising Triggering Particulates.

### 5.6. Syringeability and Extractability Resistance, and Heat Stability

In certain embodiments, the particulate and multi-particulate dosage forms of the present disclosure provide several additional abuse-deterrent properties, including syringeability resistance, extractability resistance, and heat stability. For example, the multi-particulate dosage forms resist abuse via, but not limited to, extraction of the opioid from the dosage form, syringeability of the opioid from the dosage form, and destabilization of the several abuse-deterrent attributes by various thermal pretreatment-related manipulations (e.g., heating or freezing of the dosage form before mechanical manipulations, e.g., crushing or grinding). In certain embodiments, the combination of these additional properties, along with the aforementioned resistances to crushability and grindability of the Opioid Particulates, strongly deter or prevent abuse of the inventive multi-particulate dosage form.

In certain embodiments, resistance to extractability is provided by, e.g., carbomers in the Opioid Particulates of the dosage form. In certain embodiments, carbomers (such as Carbopol 934P, Carbopol 971P, Carbopol 974P), as well as other anionic polymers that are viscosity-enhancing agents, form gel and increase viscosity in aqueous and/or alcoholic media, such as those media used by abusers attempting extraction of opioid from a given dosage form. In certain embodiments, the gelling effect of carbomers is greatly enhanced in alkaline pH resulting from the alkaline agent released from the Triggering Particulates (e.g., in attempted extraction, or in the stomach when three or more dosage units are ingested), or the alkaline agent when present in the polymer matrix. In certain embodiments, carbomers in the core form gel and further diminish drug release, e.g., permeation from the core of Opioid Particulates into the GI fluid, or into aqueous media attempting to be drawn into a syringe. In certain embodiments, polymers present in the functional coat(s), e.g., EUDRAGIT® E PO, are also involved in decreasing permeation of the opioid from the Opioid Particulates, e.g., when extraction is attempted. The alkaline agent(s) present in the dosage forms produce a rapid rise in the pH of aqueous media (e.g., in attempted extraction, or in the stomach when three or more dosage units are ingested). The polymers present in the functional coats, e.g., EUDRAGIT® E PO, become insoluble in this alkaline media; thus, the release of opioid from the dosage form is retarded.

In certain embodiments, resistance to syringeability is provided by polyoxyethylene (PEO) polymers and HPMC in the Opioid Particulates (e.g., in the core of the Opioid Particulates). The gelling characteristics of these molecules, when exposed to aqueous media, provide resistance to syringeability as the bore of the needle is blocked by the viscous nature of the diluted dosage form. In addition, carbomers included in the dosage form (e.g., in the core of the Opioid Particulates) provide further resistance to syringeability; in response to the rapidly rising pH induced by, e.g., Mg(OH)₂ in aqueous media, carbomer-based gelling is greatly enhanced, further diminishing drug release. In certain embodiments, carbomers included in the dosage form (e.g., in the core of the Opioid Particulates) provide further resistance to syringeability in response to the rising pH induced by the interaction of aqueous media with Mg(OH)₂ present in the core. Thus, less drug permeates into the aqueous media, and less drug is available to be drawn into the syringe. In certain embodiments, polymers present in the functional coats, e.g., EUDRAGIT® E PO, are also involved in resistance to syringeability. The alkaline agent(s) present in the dosage form produces a rapid rise in the pH of aqueous media. The polymers present in the functional coats, e.g., EUDRAGIT® E PO, become insoluble in this alkaline media and retard release of opioid from the dosage form. Thus, attempts to draw fluid containing the opioid into a syringe are retarded in this manner as well.

In certain embodiments, resistance to syringeability and extractability are provided by one or more properties of the dosage form. For example, resistance is provided by the gelling characteristics of polyoxyethylene (PEO) polymers and HPMC in the Opioid Particulates (e.g., in the core of the Opioid Particulates) when exposed to aqueous media; such gelling results in less drug permeating into the aqueous media, and less drug being available to be drawn into a syringe. In addition, carbomers and alkaline agent(s) included in the matrix core of the dosage form (e.g., in the core of the Opioid Particulates) provide further resistance to syringeability; in response to the rapidly rising pH induced by Mg(OH)₂ in aqueous media; carbomer-based gelling is greatly enhanced, diminishing drug release. Also, in response to the elevated pH induced by Mg(OH)₂ (present in the Triggering Particulates), the functional coat layer(s) remain relatively intact, further diminishing drug release from the dosage form. These unique combinations of elements and features of the dosage form are prominent, for example, in a physiological setting involving accidental overdose (or deliberate abuse) comprising ingestion of multiple dosage units (dosage forms).

The following examples are offered to more fully illustrate the disclosure but are not to be construed as limiting the scope thereof.

### 6. EXAMPLES

### Example 1: Crush-Resistant Oxycodone Hydrochloride Granule Cores (Opioid Granules)

Oxycodone hydrochloride granule cores were prepared for use in a 5 mg or 15 mg oxycodone hydrochloride dosage form.

TABLE 1
Formulation of Active (Opioid) Granule Cores

| | Active | Active |
|---|---|---|
| | Granules | Granules |
| | Core 1 | Core 2 |
| Components | mg/dose | mg/dose |
| Oxycodone hydrochloride | 5.00 | 15.00 |
| Polyethylene oxide (POLYOX ™) | 65.44 | 65.44 |
| Microcrystalline Cellulose (Avicel PH 101) | 10.00 | NA |
| Hypromellose (Benecel K200M) | 9.41 | 9.41 |
| Kollidon SR | 4.71 | 4.71 |
| Triethyl citrate | 3.24 | 3.24 |
| Docusate sodium (85%) with sodium | 2.00 | 2.00 |
| benzoate (15%) (DOSS) | | |
| Vitamin E (dl-α-Tocopherol) | 0.20 | 0.20 |
| Total | 100.00 | 100.00 |

### Manufacturing Procedure:

1. Oxycodone hydrochloride, polyethylene oxide, microcrystalline cellulose, hypromellose, Kollidon SR, and docusate sodium were added to a high shear granulator and mixed into a uniform powder mix using an impeller and a chopper.
2. A solution of dl-α-tocopherol solution and triethyl citrate was sprayed onto the powder mix from step #1 to achieve a uniform blend.
3. The blend from step #2 was granulated by hot-melt extrusion.
4. The granules from step #3 were processed using cryomilling to a mean particle size of about 500 μm.

### Example 2: Seal Coating of Oxycodone Hydrochloride Granule Cores

Oxycodone hydrochloride active granule cores were coated with a seal coat.

TABLE 2
Formulation of Seal Coated Granules

| | Seal coated | Seal coated |
|---|---|---|
| | granules 1 | granules 2 |
| Components | mg/dose | mg/dose |
| Active granules cores (Oxycodone | 100.00 | 100.00 |
| hydrochloride) | | |
| Hypromellose (Methocel E5 Premium LV) | 26.66 | 17.78 |
| Triethyl citrate | 2.67 | 1.78 |
| Colloidal silicon dioxide (Cab-O-Sil) | 0.67 | 0.44 |

Solvent system for coating

| | | |
|---|---|---|
| Purified water* | NA | NA |
| Dehydrated alcohol* | NA | NA |
| Total | 130.00 | 120.00 |

*Removed during process

### Coating Procedure:

1. Hypromellose was added to dehydrated alcohol in a stainless-steel container and mixed to form a uniform dispersion.
2. To the dispersion from step #1, the purified water was added and mixed until a clear solution formed.
3. To the solution from step #2, triethyl citrate was added followed by the addition of colloidal silicon dioxide and mixed to form a homogenous dispersion.
4. The granules were coated using a Wurster fluid bed coater with an inlet air temperature of 40°−50° C., and sufficient air volume for fluidization.
5. When the product temperature reached 30° C., the dispersion from step #3 was sprayed onto the granules while maintaining the product temperature of 28°−30° C. and sufficient air volume for the fluidization, until the target coating weight gain was achieved.
6. The coated granules from step #5 were dried.

### Example 3: Functional Coating of Seal Coated Oxycodone Hydrochloride Granules

Seal coated oxycodone hydrochloride granules were coated with a functional coat layer FC 1 comprising EUDRAGIT E® PO partially neutralized with succinic acid with or without cellulose acetate.

TABLE 3
Formulation of Functional Coated Active Granules (FC 1)

| | Functional | Functional | Functional | Functional | Functional | Functional |
|---|---|---|---|---|---|---|
| | Coated | Coated | Coated | Coated | Coated | Coated |
| | Granules 1 | Granules 2 | Granules 3 | Granules 4 | Granules 5 | Granules 6 |
| Components | (mg/dose) | (mg/dose) | (mg/dose) | (mg/dose) | (mg/dose) | (mg/dose) |
| Seal coated | 130.00 | 130.00 | 130.0 | 130.0 | 130.00 | 120.00 |
| granules | | | | | | |
| Amino | 92.03 | 89.70 | 33.73 | 37.37 | 32.18 | 12.00 |
| methacrylate | | | | | | |
| copolymer, NF | | | | | | |
| (EUDRAGIT ® | | | | | | |
| E PO) | | | | | | |
| Cellulose | NA | NA | 33.73 | 16.02 | 32.18 | 18.00 |
| acetate | | | | | | |
| Succinic Acid | 1.15 | 4.50 | 0.4 | 0.93 | 0.80 | NA |
| Polyethylene | 9.20 | 9.00 | NA | NA | NA | NA |
| glycol (PEG) | | | | | | |
| Talc | 13.81 | 13.40 | NA | NA | NA | NA |
| Dibutyl | NA | NA | 10.11 | 8.01 | 9.64 | 4.50 |
| Sebacate | | | | | | |
| Colloidal | 13.81 | 13.40 | 3.37 | 2.67 | 3.20 | 1.50 |
| Silicon | | | | | | |
| Dioxide | | | | | | |

Solvent system for coating

| | | | | | | |
|---|---|---|---|---|---|---|
| Acetone* | NA | NA | NA | NA | NA | NA |
| Isopropyl | NA | NA | NA | NA | NA | NA |
| alcohol* | | | | | | |
| Purified | NA | NA | NA | NA | NA | NA |
| water* | | | | | | |
| Total | 260.00 | 260.00 | 208.00 | 195.00 | 208.00 | 156.00 |

*Removed during process

### Coating Procedure:

1. To the mixture of acetone and/or isopropyl alcohol, EUDRAGIT® E PO, with or without cellulose acetate, as per granules 1-6, were added and mixed until a clear solution formed.
2. To the solution from step #1, succinic acid was added and mixed until dissolved.
3. Polyethylene glycol (PEG) solution was made by adding PEG to required quantity of water and mixed until a clear solution was formed (for granules 1 and 2).
4. To the solution from step #2, PEG solution from step #3 was added and mixed for about 10 minutes (for granules 1 and 2).
5. To the solution from step #4, talc or dibutyl sebacate, and colloidal silicon dioxide were added and mixed until a homogenous dispersion was obtained.
6. The seal coated granules were further coated using a Wurster fluid bed coater with an inlet air temperature of 30° C. and sufficient air volume for fluidization.
7. When the product temperature reached 30° C., the dispersion from step #5 was sprayed onto the seal coated granules while maintaining the product temperature of 25° C. and sufficient air volume for the fluidization, until the target coating weight gain was achieved.
8. The coated granules from step #7 were dried.

### Example 4: Second Functional Coat Layer (FC 2) of FC 1-Functional Coated Opioid Granules

FC 1 coated granules were further coated with functional coat FC 2.

TABLE 4
Formulation of Functional Coated Active Granules (FC 2)

| | Functional | Functional | Functional |
|---|---|---|---|
| | Coated | Coated | Coated |
| | Granules 7 | Granules 8 | Granules 9 |
| Components | mg/dose | mg/dose | mg/dose |
| FC 1-coated oxycodone | 208.00 | 195.00 | 156.00 |
| hydrochloride granules | | | |
| EUDRAGIT ® E PO | 59.21 | 68.42 | 72.00 |
| Succinic acid | 0.30 | 1.72 | NA |
| PEG | 5.93 | 6.84 | 7.20 |
| Colloidal silicon dioxide | 8.87 | 10.26 | NA |
| Talc | 8.87 | 10.26 | 14.40 |

Solvent system for coating

| | | | |
|---|---|---|---|
| Acetone* | NA | NA | NA |
| Isopropyl alcohol* | NA | NA | NA |
| Purified water* | NA | NA | NA |
| Total | 291.18 | 292.50 | 248.60 |

*Removed during process

### Coating Procedure:

The FC 1-coated granules were further coated with a second functional coat layer (FC 2) as follows:
1. To the mixture of acetone and isopropyl alcohol, EUDRAGIT® E PO was added and mixed until a clear solution formed.
2. To the solution from step #1, succinic acid was added and mixed until dissolved.
3. Polyethylene glycol (PEG) 6000 solution was made by adding PEG to required quantity of water and mixed until a clear solution was formed.
4. To the solution from step #2, PEG solution from step #3 was added and mixed for about 10 minutes.
5. To the solution from step #4, talc and colloidal silicon dioxide were added and mixed until a homogenous dispersion was obtained.
6. The FC 1 coated granules were further coated using a Wurster fluid bed coater with an inlet air temperature of 30° C. and sufficient air volume for fluidization.
7. When the product temperature reached 30° C., the dispersion from step #5 was sprayed onto the seal coated granules while maintaining the product temperature of 25° C. and sufficient air volume for the fluidization, until the target coating weight gain was achieved.
8. The coated granules from step #7 were dried.

### Example 5: Over Coating of Functional-Coated Oxycodone Hydrochloride Granules

Functional coated oxycodone hydrochloride granules were coated with an over coat.

TABLE 5
Formulation of Over Coated Active Granules

| | Over | Over | Over | Over |
|---|---|---|---|---|
| | Coated | Coated | Coated | Coated |
| | Granules 1 | Granules 2 | Granules 3 | Granules 4 |
| Components | (mg/dose) | (mg/dose) | mg/dose | mg/dose |
| FC 1 coated | 260.00 | 260.00 | NA | NA |
| granules | | | | |
| FC 1 + FC 2 coated | NA | NA | 291.18 | 248.60 |
| granules | | | | |
| Hypromellose, USP | 46.22 | 43.00 | 38.85 | 28.80 |
| (Methocel E5 | | | | |
| Premium LV) | | | | |
| Carbopol 971P | NA | 2.30 | NA | NA |
| Triethyl Citrate, NF | 4.62 | 4.50 | 3.88 | 3.37 |
| Colloidal Silicon | 1.16 | 2.20 | 0.97 | NA |
| Dioxide | | | | |
| Talc | NA | NA | NA | 6.27 |

Solvent system for coating

| | | | | |
|---|---|---|---|---|
| Dehydrated alcohol* | NA | NA | NA | NA |
| Purified water* | NA | NA | NA | NA |
| Total | 312.00 | 312.00 | 334.88 | 287.04 |

*Removed during process

### Coating Procedure:

1. Hypromellose was added to dehydrated alcohol in a stainless-steel container and mixed to form a uniform dispersion. In the case of granules 2, carbopol was added and mixed until it dispersed.
2. To the dispersion from step #1, purified water was added and mixed until a clear solution formed.
3. To the solution from step #2, triethyl citrate was added followed by the addition of colloidal silicon dioxide and mixed to form a homogenous dispersion.
4. The granules were coated using a Wurster fluid bed coater with an inlet air temperature of 40°−50° C., and sufficient air volume for fluidization.
5. When the product temperature reached 30° C., the dispersion from step #3 was sprayed onto the granules while maintaining the product temperature of 28°−30° C. and sufficient air volume for the fluidization, until the target coating weight gain was achieved.
6. The coated granules from step #5 were dried.

### Example 6: Triggering Granules

Triggering Granules were prepared as described.

TABLE 6
Formulation of Triggering Granules

| | Triggering | Triggering | Triggering |
|---|---|---|---|
| | Granules 1 | Granules 2 | Granules 3 |
| Components | (mg/dose) | (mg/dose) | (mg/dose) |
| Magnesium hydroxide | 250.00 | 100.00 | 135.00 |
| Mannitol | 30.40 | 16.55 | 22.50 |
| Hydroxypropyl cellulose (HPC) | 11.25 | NA | NA |
| Crospovidone (Polyplasdone XL) | 12.45 | 5.15 | 6.73 |
| Total | 304.10 | 121.70 | 164.20 |

### Manufacturing Procedure:

1. Magnesium hydroxide was added to mannitol, hydroxypropyl cellulose, and crospovidone in a high shear granulator and mixed using an impeller and chopper to achieve a uniform blend.
2. The blend from step #1 was granulated using purified water.
3. The granules from step #2 were dried at 40° C. using a forced air oven until the LOD was less than 1%.

### Example 7: Viscosity Enhancing Granules

Viscosity Enhancing Granules were prepared as described.

TABLE 7
Formulation of Viscosity Enhancing Granules

| | Viscosity Enhancing |
|---|---|
| | Granules |
| Components | (mg/dose) |
| Crospovidone, (Polyplasdone XL) | 17.50 |
| Polyethylene oxide | 31.52 |
| Hypromellose | 5.88 |
| Kollidon SR | 2.94 |
| Vitamin E (dl-α-tocopherol) | 0.13 |
| Triethyl Citrate | 2.03 |
| Docusate sodium (85%) with sodium benzoate, | 1.25 |
| (15%) | |
| Colloidal silicon dioxide | 1.25 |
| Total | 62.50 |

Seal Coat

| | |
|---|---|
| Hypromellose (Methocel E5 Premium LV) | 11.11 |
| Triethyl citrate | 1.11 |
| Colloidal silicon dioxide | 0.28 |

Solvent System for Coating

| | |
|---|---|
| Purified water | NA |
| Dehydrated alcohol | NA |
| Total | 75.00 |

### Manufacturing Procedure:

1. Polyox® was added to hypromellose, Kollidon® SR, docusate sodium, and crospovidone/starch 1500 in a high shear granulator and mixed to achieve a uniform powder mix using impeller and chopper.
2. A solution of dl-α-tocopherol solution and triethyl citrate was sprayed onto the powder mix from step #1 to achieve a uniform blend.
3. Colloidal silicon dioxide was added to the blend from step #2 and mixed to achieve a uniform blend using an impeller and chopper.
4. The blend from step #3 was granulated by hot melt extrusion, film melt, melt granulation, extrusion spheronization, or rotor or roller compactor.
5. The granules from step #4 were processed using cryomilling to a mean particle size of 500 μm.

### Seal Coating Procedure:

1. Hypromellose was added to dehydrated alcohol in a stainless-steel container and mixed to form a uniform dispersion.
2. To the dispersion from step #1, the purified water was added and mixed until a clear solution formed.
3. To the solution from step #2, triethyl citrate was added followed by the addition of colloidal silicon dioxide and mixed to form a homogenous dispersion.
4. The granules were coated using a Wurster fluid bed coater with an inlet air temperature of 40°−50° C., and sufficient air volume for fluidization.
5. When the product temperature reached 30° C., the dispersion from step #3 was sprayed onto the granules while maintaining the product temperature of 28°−30° C. and sufficient air volume for the fluidization, until the target coating weight gain was achieved.
6. The coated granules from step #5 were dried.

### Example 8: Tablet Composition

Oxycodone hydrochloride tablets (5 mg, 15 mg) were manufactured as described.

TABLE 8
Formulation Composition of Oxycodone Hydrochloride Tablets

| Components | mg/dose | mg/dose | mg/dose |
|---|---|---|---|
| Over coated oxycodone hydrochloride | 312.00 | 312.00 | 287.04 |
| active granules | | | |
| Viscosity enhancing granules | 75.00 | 75.00 | 75.00 |
| Triggering granules | 304.14 | 121.70 | 164.20 |
| Mannitol | 30.00 | 30.00 | 30.00 |
| Microcrystalline cellulose | 248.86 | 231.30 | 213.76 |
| Hydroxypropyl cellulose | 7.50 | 7.50 | 7.50 |
| Croscarmellose sodium | 18.75 | 18.75 | 18.75 |
| Magnesium stearate | 3.75 | 3.75 | 3.75 |
| Total | 1000.00 | 800.00 | 800.00 |

### Manufacturing Procedure:

1. A uniform blend of over coated active granules, viscosity enhancing granules, triggering granules, mannitol, microcrystalline cellulose, hydroxypropyl cellulose, and croscarmellose sodium was made using a V-blender.
2. To the blend from step #1, magnesium stearate was added and blended for three minutes using a V-blender.
3. The blend from step #2 was compressed into tablets using a tablet press.

### Example 9: Tablet Composition

Oxycodone hydrochloride tablets (5 mg) were manufactured as described.

TABLE 9
Formulation Composition of Oxycodone
Hydrochloride Tablets (5 mg)

| Components | mg/dose |
|---|---|
| Over coated oxycodone hydrochloride active granules | 334.88 |
| Viscosity enhancing granules | 75.00 |
| Triggering granules | 304.10 |
| Mannitol | 30.00 |
| Microcrystalline cellulose | 256.00 |
| Hydroxypropyl cellulose | 7.52 |
| Croscarmellose sodium | 18.75 |
| Magnesium stearate | 3.75 |
| Total | 1030.00 |

### Manufacturing Procedure:

1. A uniform blend of over coated active granules, viscosity enhancing granules, triggering granules, mannitol, microcrystalline cellulose, hydroxypropyl cellulose, and croscarmellose sodium was made using a V-blender.
2. To the blend from step #1, magnesium stearate was added and blended for three minutes using a V-blender.
3. The blend from step #2 was compressed into tablets using a tablet press.

### Example 10: Opioid (5 mg) Capsule Dosage Form

Capsules are filled with over coated opioid and triggering granules.

TABLE 10
Formulation Composition of Oxycodone HCl (5 mg) Capsules

| Components | mg/dose | mg/dose |
|---|---|---|
| Over coated opioid granules | 312.00 | 351.00 |
| (e.g., oxycodone hydrochloride) | | |
| Triggering granules (magnesium | 304.14 | 304.14 |
| hydroxide granules) | | |
| Total | 616.14 | 655.14 |

### Manufacturing Procedure:

1. A uniform blend of over coated opioid and triggering granules is made using a V-blender.
2. Based on the fill weight, the blend from Step #1 is filled into capsules.

### Example 11: Opioid (5 mg) Bilayer Tablet Dosage Form

Over coated opioid and triggering granules are compressed into bilayer tablets.

TABLE 11
Formulation Composition of Oxycodone
Hydrochloride (5 mg) Bilayer Tablets

| | |
|---|---|
| mg/dose | mg/dose |

Active Tablet Components

| | | |
|---|---|---|
| Over coated opioid granules | 312.00 | 351.00 |
| (e.g., oxycodone hydrochloride) | | |
| Microcrystalline cellulose | 160.21 | 171.21 |
| Hydroxypropyl cellulose | 3.75 | 3.75 |
| Croscarmellose sodium | 10.00 | 10.00 |
| Magnesium stearate | 1.50 | 1.50 |

Triggering Tablet Components

| | | |
|---|---|---|
| Triggering granules (magnesium | 304.14 | 304.14 |
| hydroxide granules) | | |
| Croscarmellose sodium | 7.00 | 7.00 |
| Magnesium stearate | 1.40 | 1.40 |
| Total | 800.00 | 850.00 |

### Manufacturing Procedure:

1. A uniform blend of over coated opioid granules, microcrystalline cellulose, hydroxypropyl cellulose, and croscarmellose sodium is made using a V-blender.
2. To the blend from step #1, magnesium stearate is added, and the mixture is further blended for 3 minutes using V-blender.
3. Similarly, a uniform blend of triggering granules is made by mixing magnesium hydroxide granules and croscarmellose sodium using a V-blender.
4. To the blend from step #3, magnesium stearate is added, and the mixture is further blended for 3 minutes using a V-blender.
5. The two blends (i.e., from step #2 and step #4) are layered on each other during compression to form bilayer tablets.

### Example 12: Opioid (5 mg) Capsule Dosage Form

Over coated opioid granules are compressed into tablets and filled into capsules along with triggering granules.

TABLE 12
Formulation Composition of Oxycodone HCl (5 mg) Capsules

| Components | mg/dose |
|---|---|
| Over coated opioid granules (e.g., oxycodone hydrochloride) | 312.00 |
| Microcrystalline cellulose | 140.86 |
| Anhydrous lactose | 100.00 |
| Hydroxypropyl cellulose | 10.00 |
| Croscarmellose sodium | 30.00 |
| Magnesium stearate | 3.00 |

External blend

| | |
|---|---|
| Triggering granules (magnesium hydroxide granules) | 304.14 |
| Total | 900.00 |

### Manufacturing Procedure:

1. A uniform blend of over coated opioid granules, microcrystalline cellulose, anhydrous lactose, hydroxypropyl cellulose, and croscarmellose sodium is made using a V-blender.
2. To the blend from step #1, magnesium stearate is added, and the mixture is further blended for 3 minutes.
3. The blend from step #2 is compressed into tablets using a tablet press.
4. The compressed tablets along with the triggering granules are filled into capsules.

### Example 13: Opioid (5 mg) Capsule Dosage Form

Over coated opioid particulates are compressed into a first tablet population. Triggering granules are compressed into a second tablet population. The two tablet populations are filled into capsules.

TABLE 13
Formulation Composition of Oxycodone
Hydrochloride (5 mg) Capsules

| | |
|---|---|
| mg/dose | mg/dose |

Active Tablet Components

| | | |
|---|---|---|
| Over coated opioid granules | 312.00 | 351.00 |
| (e.g., oxycodone hydrochloride) | | |
| Microcrystalline cellulose | 160.21 | 171.21 |
| Hydroxypropyl cellulose | 3.75 | 3.75 |
| Croscarmellose sodium | 10.00 | 10.00 |
| Magnesium stearate | 1.50 | 1.50 |

Triggering Tablet Components

| | | |
|---|---|---|
| Triggering granules (magnesium | 304.14 | 304.14 |
| hydroxide granules) | | |
| Croscarmellose sodium | 7.00 | 7.00 |
| Magnesium stearate | 1.40 | 1.40 |
| Total | 800.00 | 850.00 |

### Manufacturing Procedure:

1. A uniform blend of over coated opioid granules, microcrystalline cellulose, anhydrous lactose, hydroxypropyl cellulose, and croscarmellose sodium is made using a V-blender.
2. To the blend from step #1, magnesium stearate is added and blended for 3 minutes using V-blender and then compressed into tablets using a tablet press.
3. Similarly, a uniform blend of triggering granules is made by mixing magnesium hydroxide granules and croscarmellose sodium using a V-blender.
4. To the blend from step #3, magnesium stearate is added, and the mixture is further blended for 3 minutes using V-blender and then compressed into tablets using a tablet press.
5. Tablets from step #2 and step #4 are filled into capsules.

### Example 14: Opioid (5 mg) Capsule Dosage Form

Seal coated opioid granules are compressed into tablets, coated with cationic polymer (e.g., EUDRAGIT) partially neutralized with succinic acid, and filled into capsules along with triggering granules.

TABLE 14
Formulation Composition of Oxycodone
HCl (5 mg) capsule dosage form

| Components | mg/dose |
|---|---|
| Seal coated opioid granules (e.g., oxycodone hydrochloride) | 130.00 |
| Microcrystalline cellulose | 73.16 |
| Anhydrous lactose | 100.00 |
| Hydroxypropyl cellulose | 3.00 |
| Croscarmellose sodium | 5.00 |
| Magnesium stearate | 1.50 |

Coating

| | |
|---|---|
| EUDRAGIT ® E PO | 59.20 |
| Succinic Acid | 0.30 |
| PEG | 5.90 |
| Colloidal silicon dioxide | 8.90 |
| Talc | 8.90 |

Solvent system for coating

| | |
|---|---|
| Acetone | NA |
| Isopropyl alcohol | NA |
| Purified water | NA |

External blend

| | |
|---|---|
| Triggering granules (magnesium hydroxide granules) | 304.14 |
| Total | 700.00 |

### Manufacturing Procedure:

1. A uniform blend of seal coated opioid granules, microcrystalline cellulose, anhydrous lactose, hydroxypropyl cellulose, and croscarmellose sodium is made using a V-blender.
2. To the blend from step #1, magnesium stearate is added, and the mixture is further blended for 3 minutes using V-blender.
3. The blend from step #2 is compressed into tablets using a tablet press.
4. The compressed tablets are coated with partially neutralized EUDRAGIT and filled into capsules along with triggering granules.

### Example 15: Release Profiles of Oxycodone HCl (5 mg)

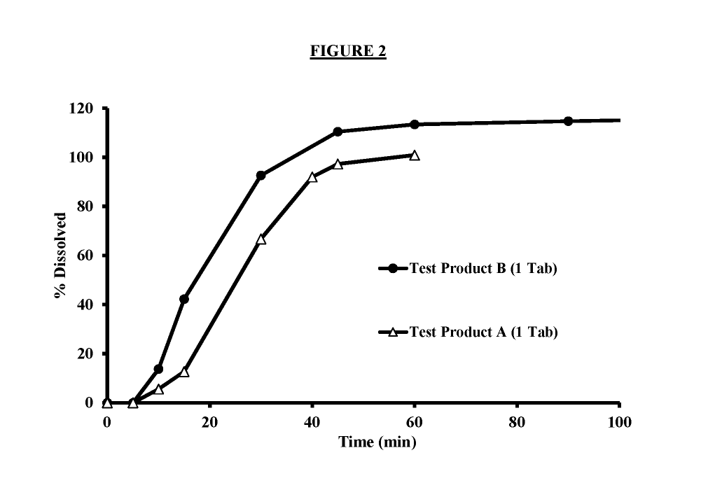

With reference to FIG. 2 for the purpose of illustration and not limitation, there is provided a graph illustrating a comparison of in vitro release profiles of oxycodone HCl tablets (5 mg) at pH 5.5. Release profiles of single oxycodone HCl tablets were measured at pH 5.5 using USP Dissolution Apparatus II (Paddle) at 50 rpm.

Test Product A tablets (A) had active pellets with a functional coat layer comprising a cationic polymer in the absence of succinic acid, whereas Test Product B tablets (•) had active pellets with a functional coat layer comprising a cationic polymer in the presence of succinic acid.

The in vitro release profiles of single oxycodone HCl tablets, as shown in FIG. 2, indicate that the immediate release of oxycodone HCl from the dosage form comprising succinic acid is food independent. The studies were conducted at pH 5.5 to investigate and address the reduction of the release rate of an active agent (e.g., an opioid) in the fed state. The release rate of Test Product B (comprising succinic acid) was markedly faster than that of Test Product A (no succinic acid), addressing the noted reduction of release rate in the fed state. Thus, succinic acid keeps the base polymer in a partially neutralized form, maintaining the immediate release properties of the dosage form, even in the fed state.

The present disclosure is well adapted to attain the ends and advantages mentioned, as well as those that are inherent therein. The embodiments disclosed above are illustrative only, as the present disclosure can be modified and practiced in different but equivalent manners apparent to those skilled in the art having the benefit of the teachings herein. Furthermore, no limitations are intended to the details of construction or design herein shown, other than as described in the claims below. It is therefore evident that the illustrative embodiments disclosed above can be altered or modified, and all such variations are considered within the scope and spirit of the present disclosure. Various publications, patents, and patent applications are cited herein, the contents of which are hereby incorporated-by-reference in their entireties.

1. A food independent, multiparticulate dosage form that provides an immediate release of an opioid when a single dosage unit is consumed intact, independent of fed or fasted state of an individual consuming the dosage form, and provides overdose protection when multiple dosage units are consumed intact, the dosage form comprising:
Active Particulates comprising a therapeutically effective amount of at least one opioid embedded in a polymer matrix, and an acid labile functional coat; and
Triggering Particulates comprising an alkaline agent;
wherein the acid labile functional coat comprises at least one functional coat layer FC 1 comprising at least one acid, a water-insoluble nonionic polymer, and a base polymer that is at least partially neutralized as a cationic salt at gastric fluid pH;
wherein the alkaline agent is present in an amount sufficient, when three or more dosage units are consumed together, to increase gastric fluid pH to a level that reduces the solubility of the acid labile functional coat and causes a decrease in the immediate release of the opioid from the dosage form to provide the overdose protection; and
wherein the acid is present in an amount that keeps the base polymer in partially neutralized form and maintains immediate release properties of the dosage form in the fed state.

2. The dosage form of claim 1, wherein the partially neutralized base polymer is a copolymer of dimethyl aminoethyl methacrylate, butyl methacrylate, and methyl methacrylate.

3. The dosage form of claim 1, wherein the acid is selected from the group consisting of succinic acid, hydrochloric acid, sulfuric acid, nitric acid, lactic acid, phosphoric acid, citric acid, acetic acid, malic acid, fumaric acid, stearic acid, tartaric acid, boric acid, benzoic acid, and mixtures thereof.

4. The dosage form of claim 1, wherein the acid is present in an amount of between about 0.1% w/w and about 5% w/w of the dosage form.

5. The dosage form of claim 4, wherein the acid is present in an amount of between about 0.1% w/w and about 0.25% w/w of the dosage form.

6. The dosage form of claim 3, wherein the acid is succinic acid.

7. The dosage form of claim 1, further comprising a second functional coat layer FC 2, completely or partially surrounding FC 1.

8. The dosage form of claim 1, wherein the water-insoluble nonionic polymer comprises cellulose acetate; cellulose acetate-based polymers; polyvinyl acetate polymers; polyvinyl acetate-based copolymers; ethylcellulose; methacrylic acid and methyl methacrylate (1:1); methacrylic acid and methyl methacrylate (1:2); copolymers of ethyl acrylate and methyl methacrylate; or mixtures thereof.

9. The dosage form of claim 8, wherein the water-insoluble nonionic polymer is cellulose acetate.

10. The dosage form of claim 1, wherein the partially neutralized base polymer and the water-insoluble nonionic polymer are present in a weight ratio of about 50:50.

11. The dosage form of claim 7, wherein FC 2 comprises an acid and a base polymer that is at least partially neutralized as a cationic salt at gastric fluid pH.

12. The dosage form of claim 11, wherein the partially neutralized base polymer of FC 2 is a copolymer of dimethylaminoethyl methacrylate, butyl methacrylate, and methyl methacrylate.

13. The dosage form of claim 1, wherein the polymer matrix comprises a nonionic polymer selected from the group consisting of a copolymer of ethyl acrylate, methyl methacrylate, and a low content of methacrylic acid ester with quaternary ammonium groups; hydroxypropylcellulose; hydroxypropyl methylcellulose; hydroxyethylcellulose; ethylcellulose; cellulose acetate butyrate; cellulose acetate; polyvinyl acetate-based polymers; polyethylene oxide polymers; and mixtures thereof.

14. The dosage form of claim 13, wherein the nonionic polymer is a mixture of a polyethylene oxide polymer, hydroxypropyl methylcellulose, and a polyvinyl acetate-based polymer.

15. The dosage form of claim 13, wherein the nonionic polymer is a mixture of a polyethylene oxide polymer and hydroxypropyl methylcellulose.

16. The dosage form of claim 1, wherein the alkaline agent present in the Triggering Particulates is selected from the group consisting of aluminum hydroxide, sodium hydroxide, potassium hydroxide, calcium hydroxide, magnesium hydroxide, calcium carbonate, sodium carbonate, potassium bicarbonate, sodium bicarbonate, ammonia, tertiary sodium phosphate, diethanolamine, ethylenediamine, N-methylglucamine, L-lysine, and mixtures thereof.

17. The dosage form of claim 16, wherein the alkaline agent is magnesium hydroxide.

18. The dosage form of claim 1, wherein the alkaline agent is present in an amount of up to about 40% w/w of the total weight of the dosage form.

19. The dosage form of claim 18, wherein the alkaline agent is present in an amount of from about 25% w/w to about 32% w/w of the total weight of the dosage form.

20. The dosage form of claim 1, wherein the Active Particulates further comprise a plasticizer in an amount sufficient to enhance elasticity and crush resistance of the polymer matrix.

21. The dosage form of claim 20, wherein the crush resistance of the polymer matrix is enhanced to an extent that prevents reducing particulates to a size that can be insufflated.

22. The dosage form of claim 20, wherein the plasticizer acts as one or more of an aversion agent and a tissue irritant.

23. The dosage form of claim 20, wherein the plasticizer is selected from the group consisting of triethyl citrate, propylene glycol, polyethylene glycols, triacetin, diethylene glycol monoethyl ether, dibutyl sebacate, diethyl phthalate, and mixtures thereof.

24. The dosage form of claim 1, wherein the Active Particulates further comprise one or more of a surfactant and a viscosity enhancing agent.

25. The dosage form of claim 1, wherein the opioid is selected from the group consisting of oxycodone, hydrocodone, oxymorphone, and hydromorphone, and pharmaceutically acceptable salts thereof.

26. A method of treating pain comprising administering to a patient in need thereof a food independent, multiparticulate dosage form that provides an immediate release of an opioid when a single dosage unit is consumed intact, independent of fed or fasted state of an individual consuming the dosage form, and provides overdose protection when multiple dosage units are consumed intact, the dosage form comprising:
Active Particulates comprising a therapeutically effective amount of at least one opioid embedded in a polymer matrix, and an acid labile functional coat; and
Triggering Particulates comprising an alkaline agent;
wherein the acid labile functional coat comprises at least one functional coat layer FC 1 comprising at least one acid, a water-insoluble nonionic polymer, and a base polymer that is at least partially neutralized as a cationic salt at gastric fluid pH;
wherein the alkaline agent is present in an amount sufficient, when three or more dosage units are consumed together, to increase gastric fluid pH to a level that reduces the solubility of the acid labile functional coat and causes a decrease in the immediate release of the opioid from the dosage form to provide the overdose protection; and
wherein the acid is present in an amount that keeps the base polymer in partially neutralized form and maintains immediate release properties of the dosage form in the fed state.

27. A method of making a food independent, multiparticulate dosage form that provides an immediate release of an opioid when a single dosage unit is consumed intact, independent of fed or fasted state of an individual consuming the dosage form, and provides overdose protection when multiple dosage units are consumed intact, the method comprising:
making Active Particulates by hot-melt extruding a blend of oxycodone hydrochloride, polyethylene oxide, and at least one additional water-soluble nonionic polymer, and coating the extrudates with an acid labile functional coat comprising at least one functional coat layer FC 1 comprising at least one acid, a water-insoluble nonionic polymer, and a base polymer that is at least partially neutralized as a cationic salt at gastric fluid pH;
making Triggering Particulates comprising an alkaline agent;
mixing the Active Particulates and the Triggering Particulates into a uniform blend;
mixing the blend with magnesium stearate; and compressing the mixture into a tablet.
